(19) United States
(12) Patent Application Publication
(10) Pub. No.: US 2023/0364061 A1
(43) Pub. Date: Nov. 16, 2023

(54) THIAZOLIDINE DERIVATIVES FOR INHIBITING CAV3.2-USP5 INTERACTIONS

(71) Applicant: Zymedyne Therapeutics Inc., Calgary (CA)

(72) Inventor: Gerald Werner Zamponi, Calgary (CA)

(21) Appl. No.: 18/027,222

(22) PCT Filed: Sep. 23, 2021
(86) PCT No.: PCT/CA2021/051325

§ 371 Date: Mar. 20, 2023

Related U.S. Application Data
(60) Provisional application No. 63/082,231, filed on Sep. 23, 2020.

(51) Int. Cl. A61K 31/426; A61P 25/04

(52) U.S. Cl. CPC A61K 31/426; A61P 25/04

(57) ABSTRACT

The present application relates to various uses of thiazolidine derivatives of compounds of Formula II in the treatment of diseases, disorders or conditions that are treatable by inhibiting interactions between Cav3.2 T-type calcium channels (Cav3.2) and ubiquitin specific peptidase 5 (USPS). For example, the one or more compounds of Formula II are useful as an analgesic in the treatment of pain. The present application also relates to compounds of Formula I or pharmaceutically acceptable salts, solvates and/or prodrugs thereof. (I) (II)

### RELATED APPLICATIONS

The present application claims the benefit of priority of co-pending U.S. provisional patent application no. 63/082,231 filed on Sep. 23, 2020, the contents of which are incorporated herein by reference in their entirety.

### FIELD

The present application relates to substituted thiazolidine compounds to compositions comprising them, and to their use in therapy. More particularly, it relates to thiazolidine derivatives useful in the treatment of diseases, disorders or conditions treatable by inhibiting interactions between Cav3.2 voltage gated T-type calcium channel and ubiquitin specific peptidase 5 (USP5).

### BACKGROUND

In the primary afferent pain pathway, voltage gated T-type calcium channels sustain neuronal firing and appear to contribute to neurotransmitter release at afferent terminals in the spinal dorsal horn. The prominent T-type channel subtype expressed in peripheral afferents is Cav3.2. Cav3.2 T-type calcium channels are important mediators of pain signaling and their activity is upregulated in states of chronic pain (FIG. 1)¹. Conversely inhibiting these ion channels mediates analgesia in preclinical models. It has been discovered that the aberrant upregulation of Cav3.2 is due to an injury induced increase in expression of the enzyme USP5, which associates with these channels and increases their stability²⁻⁹. This leads to more Cav3.2 channels in pain sensing neurons and thus more pain. It has been shown that depleting sensory neurons of Cav3.2, or preventing the interaction between USP5 and the channels by using cell permeant decoy peptides protects from inflammatory, neuropathic, diabetic and post-surgical pain in mice (FIG. 2)⁹. A high throughput ELISA screen has been used to identify small organic molecules that could prevent USP5 interactions with the channels⁷(see FIG. 3). An initial screen of an active compound library at the Centre for Drug research and Development (CDRD at the University of British Columbia) yielded two hits (suramin and gossypetin) that were found to protect from diabetic, visceral, inflammatory and neuropathic pain in mice. Therefore, inhibiting the interaction between Cav3.2 and USP5 may be a potential target for novel therapeutics.

U.S. Pat. No. 9,993,522 discloses the use of peptide inhibitor of the interaction between Cav3.2 and USP5 for inhibiting a Cav3.2 channel function.

SUMMARY

Compounds have been identified which are capable of inhibiting or blocking the interaction of Cav3.2 T-type calcium channels (Cav3.2) and ubiquitin specific peptidase 5 (USP5).

Accordingly, the present application includes a method for inhibiting interactions between Cav3.2 T-type calcium channels (Cav3.2) and ubiquitin specific peptidase 5 (USP5) in a cell, either in a biological sample or in a patient, comprising administering an effective amount of one or more compounds of Formula II or a pharmaceutically acceptable salt and/or solvate thereof to the cell,

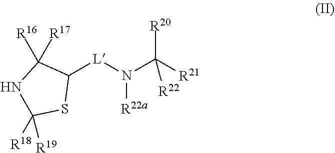

wherein:
R¹⁶ and R¹⁸ are independently selected from H, halo, CN, NO₂, C₁₋₆alkyl, C₁₋₆haloalkyl, OR²³, SR²³, NR²⁴R²⁶, C(O)R²³, CO₂R²³, CONR²⁴R²⁶, SOR²³ SO₂R²³, and SO₂NR²⁴R²⁶;
R¹⁷ and R¹⁹ are independently selected from H and C₁₋₆alkyl; or
R¹⁶ and R¹⁷ are joined together to form ═X³, and/or R¹⁸ and R¹⁹ are joined together to form ═X⁴;
X³ and X⁴ are independently selected from O and S;

### L′ is C₁₋₄alkylene;

R²⁰ and R²¹ are independently selected from H, halo, CHO, CN, NO₂, OH, SH, NH₂, CO₂H, C(O)NH₂, POSH, SO₂H, SO₂NH₂, NHSO₂H, C₁₋₁₀alkyl, C₂₋₁₀alkenyl, C₂₋₁₀alkynyl, C₃₋₁₆cycloalkyl, C₃₋₁₆heterocycloalkyl, C₆₋₁₀aryl, C₅₋₁₀heteroaryl, C₁₋₁₀alkyleneC₃₋₁₆cycloalkyl, C₁₋₁₀alkyleneC₃₋₁₆heterocycloalkyl, C₁₋₁₀alkyleneC₋₁₀aryl, C₁₋₁₀alkyleneC₅₋₁₆heteroaryl, OC₁₋₁₀alkyl, SC₁₋₁₀alkyl, NH(C₁₋₁₀alkyl), N(C₁₋₁₀alkyl)(C₁₋₁₀alkyl), C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), PO(OC₁₋₁₀alkyl)(OC₁₋₁₀alkyl), SO₂C₁₋₁₀alkyl, SO₂NH(C₁₋₁₀oalkyl), SO₂N(C₁₋₁₀alkyl)(C₁₋₁₀alkyl), NC₁₋₁₀alkylSO₂(C₁₋₁₀alkyl), and NHSO₂(C₁₋₁₀alkyl), provided R²⁰ and R²¹ are not both H, or
R²⁰ and R²¹ together with the carbon to which they are attached form C₃₋₈cycloalkyl, and each alkyl, alkenyl, alkynyl, cycloalkyl, heterocycloalkyl, aryl, heteroaryl and alkylene group in R²⁰ and R²¹ is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁰), SOR₂₆ SO₂R²⁶, SO₂NR²⁷R²⁸, and NR²⁷SO₂R²⁶;
R²² is selected from H, C₁₋₆alkyl and C₁₋₆alkyl substituted with one or more substituents selected from halo, NH₂, OH, SH, SC₁₋₆alkyl, OC₁₋₆alkyl, NH(C₁₋₆alkyl), N(C₁₋₆alkyl)₂, C(O)H, C(O)C₁₋₆alkyl, CO₂H, C(O)₂C₁₋₆alkyl, CONH₂, CONHC₁₋₆alkyl, CON(C₁₋₆alkyl)₂;
R^{22a} is selected from H, C₁₋₆alkyl and C₁₋₆alkyl substituted with one or more substituents selected from halo, NH₂, OH, SH, SC₁₋₆alkyl, OC₁₋₆alkyl, NH(C₁₋₆alkyl), N(C₁₋₆alkyl)₂, C(O)H, C(O)C₁₋₆alkyl, CO₂H, C(O)₂C₁₋₆alkyl, CONH₂, CONHC₁₋₆alkyl, CON(C₁₋₆alkyl)₂;
R²³, R²⁴, and R²⁵ are independently selected from H, C₁₋₆alkyl, C₂₋₆alkenyl and C₂₋₆alkynyl;
R²⁶, R²⁷, R²⁸, R²⁹, and R³⁰ are independently selected from H and C₁₋₆alkyl; and each alkyl, alkylene, alkenyl and alkynyl is optionally fluorosubstituted.

The present application also includes a method of treating a disease, disorder or condition that is treatable by inhibiting interactions between Cav3.2 and USP5 comprising administering a therapeutically effective amount of one or more compounds of Formula II or a pharmaceutically acceptable salt and/or solvate thereof to a subject in need thereof.

The present application also includes a method for inhibiting Cav3.2 deubiquitination in a cell, either in a biological sample or in a patient, comprising administering an effective amount of one or more compounds of Formula II or a pharmaceutically acceptable salt and/or solvate thereof to the cell.

The present application also includes a method of treating a disease, disorder or condition that is treatable by inhibiting Cav3.2 deubiquitination comprising administering a therapeutically effective amount of one or more compounds of Formula II or a pharmaceutically acceptable salt and/or solvate thereof to a subject in need thereof.

In an embodiment, the disease, disorder or condition treatable by inhibiting interactions between Cav3.2 and USP5 or inhibiting Cav3.2 deubiquitination is pain.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF DRAWINGS

The embodiments of the application will now be described in greater detail with reference to the attached drawings in which:

FIG. 1 is a schematic illustrating the role of Cav3.2 voltage gated T-type calcium channels in the primary effect pain pathway.

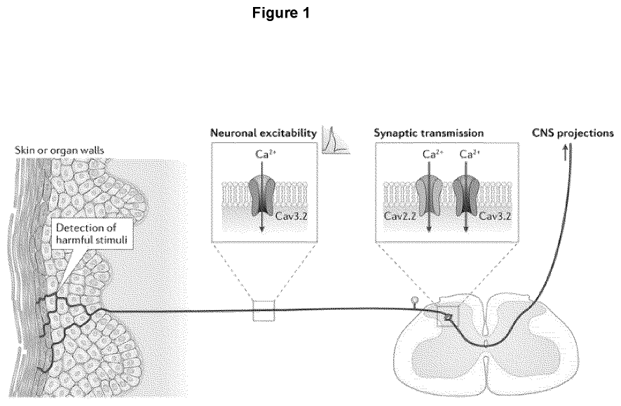

FIG. 2 is a schematic illustrating the concept of inhibiting Cav3.2 protein interactions with the deubiquitinase USP5.

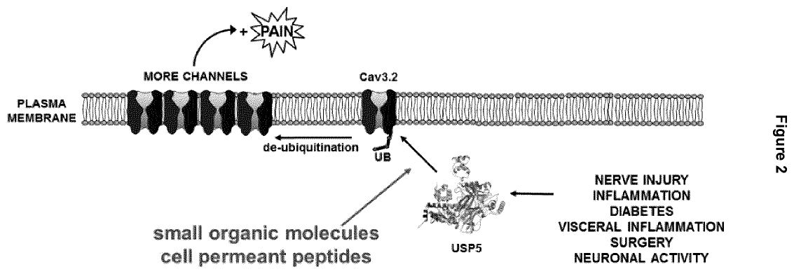

FIG. 3 is a schematic of the high throughput ELISA assay used to screen the exemplary compound of the applications. Here the Cav3.2 III-1V linker is immobilized and then recombinant USP5 is allowed to bind. Binding is detected via antibodies conjugated to horseradish peroxidase for a fluorescent readout. Compounds are applied and a reduction in fluorescence is indicative of a disruption of USP5-Cav3.2 interactions.

FIG. 4 is a graph showing the results of the of immunoprecipitation assay with Cav3.2 channels and a rabbit 3.2 polyclonal antibody using whole brain lysates and probed for USP5 (n=5). Quantifications were normalized to bound USP5 to Cav3.2 IP/alpha-tubulin. 1 uM of exemplary compounds II-1 and II-2 were used in the incubation. 5 uM of a positive control compound was used in the incubation. These data show that II-1 and II-2 disrupt the association of USP5 and Cav3.2 channels in neuronal tissue.

FIG. 5 shows the effect of exemplary compound II-1 on formalin induced nocifensive responses in phase I (left graph) and II (right graph) in mice. Formalin was injected into the hindpaw of a mouse, and the time that the mouse spent licking and biting its paw was determined. There are two phases of these nocifensive behavioral responses—phase I occurs immediately after formalin injection and reflects an acute response, phase II occurs approximately thirty minutes after formalin injection and reflects inflammatory pain. II-1 inhibited the duration of both phases in a dose dependent manner.

FIG. 6 shows the effect of exemplary compound II-1 on Complete Freund's Adjuvant (CFA) induced thermal hypersensitivity in mice. Here CFA was injected into the hindpaw and this results in chronic pain hypersensitivity, which is reflected in greater sensitivity the thermal stimuli. The latency to paw withdrawal in response to radiate heat to the paw decreases after CFA, and II-1 reverses this hypersensitivity in a dose dependent manner, indicating a strong analgesic effect of II-1.

FIG. 7 shows the effects of exemplary compound II-1 on CFA mediated thermal hypersensitivity in wild type (CaV3.2^{+/+}) mice and in Cv3.2 knockout (Cav3.2-'-) mice. The experiments show that in Cav3.2 knockout mice, exemplary compound II-1 is no longer effective in reversing CFA-induced thermal hypersensitivity.

FIG. 8 examines the effect of exemplary compound II-1 on in vitro deubiqutinase activity of USP5. Here recombinant USP5 (either the long or the short splice variant) is incubated with a ubiquitinated substrate, and deubiquitination is measured via a fluorescence readout as a function of time. The commercially available deubiquitinase inhibitor Degrasyn™ inhibits this fluorescence signal. Exemplary compounds II-1 and II-2 (not shown) fail to inhibit deubiquitination. These data thus show that the observed analgesic effects of exemplary compound II-1 are not indirectly due to an effect in the enzymatic activity of USP5, but instead due to inhibiting the interaction with Cav3.2 (see FIG. 4).

FIG. 9 shows a dose-response curve of exemplary compound II-1 on the percent inhibition in the Cav3.2-USP5 ELISA assay of Example 1.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

The term “compound(s) of the application” or “compound(s) of the present application” and the like as used herein refers to a compound of Formula I and/or Formula II or pharmaceutically acceptable salts and/or solvates thereof.

The term “composition(s) of the application” or “composition(s) of the present application” and the like as used herein refers to a composition, such a pharmaceutical composition, comprising one or more compounds of the application.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the application exist as individual salts and hydrates, as well as a combination of, for example, a solvate of a salt of a compound of the application.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The present description refers to a number of chemical terms and abbreviations used by those skilled in the art. Nevertheless, definitions of selected terms are provided for clarity and consistency.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀ alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms.

The term “cycloalkyl,” as used herein, whether it is used alone or as part of another group, refers to cyclic groups containing from 3 to 20 atoms and at least one carbocyclic non aromatic ring. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “heterocycloalkyl” as used herein, whether it is used alone or as part of another group, refers to cyclic groups containing at least one non-aromatic ring containing from 3 to 20 atoms in which one or more of the atoms are a heteroatom selected from O, S and N and the remaining atoms are C. Heterocycloalkyl groups are either saturated or unsaturated (i.e., contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as selected from O, S and N and the remaining atoms are C. Heterocycloalkyl groups are optionally benzofused.

The term “aryl” as used herein, whether it is used alone or as part of another group, refers to carbocyclic groups containing at least one aromatic ring and contains either 6, 9 or 10 carbon atoms, such as phenyl, indanyl or naphthyl.

The term “heteroaryl” as used herein, whether it is used alone or as part of another group, refers to cyclic groups containing at least one heteroaromatic ring containing 5-20 atoms in which one or more of the atoms are a heteroatom selected from O, S and N and the remaining atoms are C. When a heteroaryl group contains the prefix this prefix indicates the number of carbon atoms in the corresponding carbocyclic group, in which one or more, suitably 1 to 5, of the ring atoms is replaced with a heteroatom as defined above. Heteroaryl groups are optionally benzofused.

All cyclic groups, including aryl, heteroaryl, heterocycloalkyl and cycloalkyl groups, contain one or more than one ring (i.e., are polycyclic). When a cyclic group contains more than one ring, the rings may be fused, bridged, spirofused or linked by a bond.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “fluorosubstituted” refers to the substitution of one or more, including all, available hydrogens in a referenced group with fluoro.

The terms “halo” or “halogen” as used herein, whether it is used alone or as part of another group, refers to a halogen atom and includes fluoro, chloro, bromo and iodo.

The term “substituted” as used herein means that the referenced atom contains at least one substituent group other that a hydrogen atom.

The term “substituent” as used herein refers to any chemical grouping, including groups comprising carbon atoms and/or heteroatoms that is compatible with the reaction conditions of the processes of the application.

In the processes of the application, it is typical for the compounds, including starting materials and products to be present as a mixture of isomers. For example, when it is shown that the R- or S-isomer is a product or starting material of a reaction, this means that that isomer is present in greater than 80%, 85%, 90%, 95%, 98% or 99% by weight based on the total amount of R- and S-isomers.

The term “solvent” includes both a single solvent and a mixture “comprising two or more solvents.

The term “available”, as in “available hydrogen atoms” or “available atoms” refers to atoms that would be known to a person skilled in the art to be capable of replacement by a substituent.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3^{rd} Edition, 2003, Georg Thieme Verlag (The Americas).

The products of the processes of the application may be isolated according to known methods, for example, the compounds may be isolated by evaporation of the solvent, by filtration, centrifugation, chromatography or other suitable method.

One skilled in the art will recognize that where a reaction step of the present application is carried out in a variety of solvents or solvent systems, said reaction step may also be carried out in a mixture of the suitable solvents or solvent systems.

The term “cell” as used herein refers to a single cell or a plurality of cells and includes a cell either in a cell culture or in a subject.

The term “subject” as used herein includes all members of the animal kingdom including mammals, and suitably refers to humans. Thus, the methods and uses of the present application are applicable to both human therapy and veterinary applications.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvates” as used herein refers to complexes formed between a compound and a solvent from which the compound is precipitated or in which the compound is made. The term “solvate” as used herein means a compound, or a salt of a compound, wherein molecules of a suitable solvent are incorporated in the crystal lattice.

The term “prodrug” as used herein means a compound, or salt and/or solvate of a compound, that, after administration, is converted into an active drug.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e., not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. Treatment methods comprise administering to a subject a therapeutically effective amount of one or more of the compounds of the application and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition treatable by inhibiting interactions between Cav3.2 T-type calcium channels (Cav3.2) and ubiquitin specific peptidase 5 (USP5) in a cell, or manifesting a symptom associated with a disease, disorder or condition treatable by inhibiting interactions between Cav3.2 T-type calcium channels (Cav3.2) and ubiquitin specific peptidase 5 (USP5) in a cell.

As used herein, the term “effective amount” or “therapeutically effective amount” means an amount of a compound, or one or more compounds, of the application that is effective, at dosages and for periods of time necessary to achieve the desired result. For example, in the context of treating a disease, disorder or condition treatable by inhibiting interactions between Cav3.2 T-type calcium channels (Cav3.2) and ubiquitin specific peptidase 5 (USP5), an effective amount is an amount that, for example, inhibits interactions between Cav3.2 T-type calcium channels (Cav3.2) and ubiquitin specific peptidase 5 (USP5) compared to the inhibition without administration of the one or more compounds. Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom. When the disease is pain, amounts that are effective can cause a reduction, for example, in the sensation and/or duration of pain.

The expression “inhibiting interactions between Cav3.2 T-type calcium channels (Cav3.2) and ubiquitin specific peptidase 5 (USP5)” or “ inhibiting interactions between Cav3.2 and USP5” as used herein refers to inhibiting, blocking and/or disrupting an interaction between a therapeutically relevant binding partner, such as USP5, with the Cav3.2 binding domain in a cell. The inhibiting, blocking and/or disrupting causes a therapeutic effect in the cell.

The expression “inhibiting Cav3.2 deubiquitination” as used herein refers to inhibiting, blocking and/or disrupting the removal of ubiquitin from ubiquitin-conjugated Cav3.2. The inhibiting, blocking and/or disrupting the removal of ubiquitin causes a therapeutic effect in the cell.

By “inhibiting, blocking and/or disrupting” it is meant any detectable inhibition, block and/or disruption in the presence of a compound compared to otherwise the same conditions, except for in the absence in the compound.

The term “Cav3.2” as used herein is an isoform of T-type calcium channels which contributes to propagation and transmission of nociceptive information in the afferent pain pathway.

The term “USP5” or “ubiquitin specific peptidase 5”, as used herein refers to a deubiquitinating enzyme responsible for removing ubiquitin groups from ubiquitin-conjugated proteins.

The term “inhibit” or “inhibition” as used herein refers to any decrease in an interactions between Cav3.2 T-type calcium channels (Cav3.2) and ubiquitin specific peptidase 5 (USP5) in the presence of one or more compounds of the application compared to a control (for example, otherwise identical conditions except for the absence of one of more compounds of the application).

The term “administered” as used herein means administration of a therapeutically effective amount of one or more compounds or compositions of the application to a cell, tissue, organ or subject.

The term “drug” as used herein, is intended to refer to any compound or mixture of compounds which is capable of exerting an effective pharmacological effect.

### II. Methods and Uses of the Application

The Applicants have identified a family of compounds which are capable of inhibiting and/or blocking Cav3.2 T-type calcium channel (Cav3.2) function and/or expression levels by inhibiting, blocking and/or disrupting interactions between Cav3.2 and ubiquitin specific peptidase 5 (USP5). In an embodiment, the compounds have been shown to inhibit, block and/or disrupt interactions between Cav3.2 and ubiquitin specific peptidase 5 (USP5). Therefore, the compounds may be useful for inhibiting, blocking and/or disrupting Cav3.2 deubiquitination.

Accordingly, the present application includes a method for inhibiting interactions between Cav3.2 T-type calcium channels (Cav3.2) and ubiquitin specific peptidase 5 (USP5) in a cell, either in a biological sample or in a patient, comprising administering an effective amount of one or more compounds of Formula II or a pharmaceutically acceptable salt and/or solvate thereof to the cell,

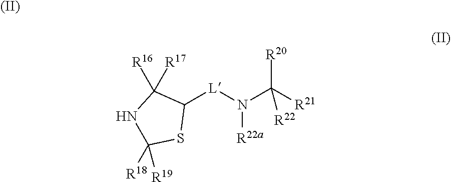

wherein
R¹⁶ and R¹⁸ are independently selected from H, halo, CN, NO₂, C₁₋₆alkyl, C₁₋₆haloalkyl, OR²³, SR²³, NR²⁴R²⁵, C(O)R²³, CO₂R²³, CONR²⁴R²⁵, SOR²³, SO₂R²³, and SO₂NR²⁴R²⁵;
R¹⁷ and R¹⁹ are independently selected from H and C₁₋₆alkyl; or
R¹⁶ and R¹⁷ are joined together to form =X³, and/or R¹⁸ and R¹⁹ are joined together to form =X⁴;
X³ and X⁴ are independently selected from O and S;

### L′ is C₁₋₄alkylene;

R²⁰ and R²¹ are independently selected from H, halo, CHO, CN, NO₂, OH, SH, NH₂, CO₂H, C(O)NH₂, POSH, SO₂H, SO₂NH₂, NHSO₂H, C₁₋₁₀alkyl, C₂₋₁₀alkenyl, C₂₋₁₀alkynyl, C₃₋₁₆cycloalkyl, C₃₋₁₆heterocycloalkyl, C₆₋₁₀aryl, C₅₋₁₀heteroaryl, C₁₋₁₀alkyleneC₃₋₁₆cycloalkyl, C₁₋₁₀alkyleneC₃₋₁₆heterocycloalkyl, C₁₋₁₀alkyleneC₆₋₁₀aryl, C₁₋₁₀alkyleneC₅₋₁₆heteroaryl, OC₁₋₁₀alkyl, SC₁₋₁₀alkyl, NH(C₁₋₁₀alkyl), N(C₁₋₁₀alkyl)(C₁₋₁₀alkyl), C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), PO(OC₁₋₁₀alkyl)(OC₁₋₁₀alkyl), SO₂C₁₋₁₀alkyl, SO₂NH(C₁₋₁₀alkyl), SO₂N(C₁₋₁₀alkyl)(C₁₋₁₀alkyl), NC₁₋₁₀alkylSO₂(C₁₋₁₀alkyl), and NHSO₂(C₁₋₁₀alkyl) provided R²⁰ and R²¹ are not both H, or
R²⁰ and R²¹ together with the carbon to which they are attached form C3_8cycloalkyl, and each alkyl, alkenyl, alkynyl, cycloalkyl, heterocycloalkyl, aryl, heteroaryl and alkylene group in R²⁰ and R²¹ is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁰), SOR²⁶ SO₂R²⁶, SO₂NR²⁷R²⁸, and NR²⁷SO₂R²⁶;
R²² is selected from H, C₁₋₆alkyl and C₁₋₆alkyl substituted with one or more substituents selected from halo, NH₂, OH, SH, SC₁₋₆alkyl, OC₁₋₆alkyl, NH(C₁₋₆alkyl), N(C₁₋₆alkyl)₂, C(O)H, C(O)C₁₋₆alkyl, CO₂H, C(O)₂C₁₋₆alkyl, CONH₂, CONHC₁₋₆alkyl, CON(C₁₋₆alkyl)₂;
R^{22a} is selected from H, C₁₋₆alkyl and C₁₋₆alkyl substituted with one or more substituents selected from halo, NH₂, OH, SH, SC₁₋₆alkyl, OC₁₋₆l alkyl, NH(C₁₋₆alkyl), N(C₁₋₆alkyl)₂, C(O)H, C(O)C₁₋₆alkyl, CO₂H, C(O)₂C₁₋₆alkyl, CONH₂, CONHC₁₋₆alkyl, CON(C₁₋₆alkyl)₂;

R²³, R²⁴, and R²⁵ are independently selected from H, C₁₋₆alkyl, C₂₋₆alkenyl and C₂₋₆alkynyl;

R²⁶, R²⁷, R²⁸, R²⁹, and R³⁰ are independently selected from H and C₁₋₆alkyl; and

each alkyl, alkylene, alkenyl and alkynyl is optionally fluorosubstituted.

The present application includes a method for inhibiting interactions between Cav3.2 T-type calcium channels (Cav3.2) and ubiquitin specific peptidase 5 (USPS) in a cell, either in a biological sample or in a patient, comprising administering an effective amount of one or more compounds of Formula II or a pharmaceutically acceptable salt and/or solvate thereof to the cell,

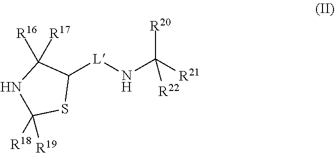

wherein
R¹⁶ and R¹⁸ are independently selected from H, halo, CN, NO₂, C₁₋₆alkyl, C₁₋₆haloalkyl, OR²³, SR²³, NR²⁴R²⁵, C(O)R²³, CO₂R²³, CONR²⁴R²⁵, SOR²³, SO₂R²³, and SO₂NR²⁴R²⁵;
R¹⁷ and R¹⁹ are independently selected from H and C₁₋₆alkyl; or
R¹⁶ and R¹⁷ are joined together to form =X³, and/or R¹⁸ and R¹⁹ are joined together to form =X⁴;
X³ and X⁴ are independently selected from O and S;

### L′ is C₁₋₄alkylene;

R²⁰ and R²¹ are independently selected from halo, CHO, CN, NO₂, OH, SH, NH₂, CO₂H, C(O)NH₂, POSH, SO₂H, SO₂NH₂, NHSO₂H, C₁₋₁₀alkyl, C₂₋₁₀alkenyl, C₂₋₁₀alkynyl, C₃₋₁₆cycloalkyl, C₃₋₁₆heterocycloalkyl, C₆₋₁₀aryl, C₅₋₁₀heteroaryl, C₁₋₁₀alkyleneC₃₋₁₆cycloalkyl, C₁₋₁₀alkyleneC₃₋₁₆heterocycloalkyl, C₁₋₁₀alkyleneC₆₋₁₀aryl, C₁₋₁₀alkyleneC₆₋₁₆heteroaryl, OC₁₋₁₀alkyl, SC₁₋₁₀alkyl, NH(C₁₋₁₀alkyl), N(C₁₋₁₀alkyl)(C₁₋₁₀alkyl), C(O)C₁₋₁₀alkyl, CO₂C₁₋₁₀alkyl, C(O)NHC₁₋₁₀ioalkyl, C(O)N(C₁₋₁₀alkyl)(C₁₋₁₀alkyl), PO(OC₁₋₁₀alkyl)(OC₁₋₁₀alkyl), SO₂C₁₋₁₀alkyl, SO₂NH(C₁₋₁₀alkyl), SO₂N(C₁₋₁₀alkyl)(C₁₋₁₀alkyl), NC₁₋₁₀alkylSO₂(C₁₋₁₀alkyl), and NHSO₂(C₁₋₁₀alkyl),
and each alkyl, alkenyl, alkynyl, cycloalkyl, heterocycloalkyl, aryl, heteroaryl and alkylene group in R²⁰ and R²¹ is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁰), SOR²⁶, SO₂R²⁶, SO₂NR²⁷R²⁸, and NR²⁷SO₂R²⁶;
R²² is selected from H and C₁₋₆alkyl;
R²³, R²⁴, and R²⁵ are independently selected from H, C₁₋₆alkyl, C₂₋₆alkenyl and C₂₋₆alkynyl;
R²⁶, R²⁷, R²⁸, R²⁹, and R³⁰ are independently selected from H and C₁₋₆alkyl; and
each alkyl, alkylene, alkenyl and alkynyl is optionally fluorosubstituted.

In an embodiment, R¹⁶ and R¹⁸ are independently selected from H, halo, CN, NO₂, C₁₋₆alkyl, C₁₋₆haloalkyl, OR²³, SR²³, NR²⁴R²⁵, C(O)R²³, CO₂R²³, CONR²⁴R²⁵, SOR²³, SO₂R²³, and SO₂NR²⁴R²⁵, and each alkyl, alkylene, alkenyl and alkynyl is optionally fluorosubstituted. In an embodiment, R¹⁶ and R¹⁸ are independently selected from H, F, Cl, CN, NO₂, C₁₋₃alkyl, C₁₋₃haloalkyl, OR²³, SR²³, NR²⁴R²⁵, C(O)R²³, CO₂R²³, CONR²⁴R²⁵, SOR²³ SO₂R²³, and SO₂NR²⁴R²⁵, and each alkyl, alkylene, alkenyl and alkynyl is optionally fluorosubstituted. In an embodiment, R¹⁶ and R¹⁸ are independently selected from H, F, Cl, CN, NO₂, C₁₋₃alkyl, C₁₋₃haloalkyl, OR²³, SR²³, NR²⁴R²⁵, C(O)R²³, CO₂R²³, C(O)NR²⁴R²⁵, SOR²³ SO₂R²³, and SO₂NR²⁴R²⁵ and each alkyl, alkylene, alkenyl and alkynyl is optionally fluorosubstituted. In an embodiment, R²³, R²⁴, and R²⁵ are independently selected from H, C₁₋₄alkyl, C₂₋₆alkenyl and C₂₋₆alkynyl, and each alkyl, alkylene, alkenyl and alkynyl is optionally fluorosubstituted. In an embodiment, R²³, R²⁴, and R²⁵ are independently selected from H and C₁₋₄alkyl and alkyl is optionally fluorosubstituted. In an embodiment, R¹⁶ and R¹⁸ are independently selected from H, F, Cl, CN, NO₂, CH₃, CF₃, CCl₃, CH₂CH₂F, CH₂CF₃, OH, OCH₃, SH, SCH₃, NH₂, NHCH₃, N(CH₃)₂, CHO, C(O)CH₃, CO₂H, CO₂CH₃, CONH₂, CON(CH₃)₂, CONHCH₃, SOCH₃, SO₂H, SO₂CH₃, SO₂N(CH₃)₂, SO₂NHCH₃, and SO₂N(CH₃)₂. In an embodiment, R¹⁶ and R¹⁸ are independently selected from H, F, Cl, CN, NO₂, CH₃, CF₃, CCl₃, CH₂CH₂F, CH₂CF₃, OH, OCH₃, SH, SCH₃, NH₂, NHCH₃, N(CH₃)₂, CHO, and C(O)CH₃. In an embodiment, R¹⁶ and R¹⁸ are independently selected from H, F, CN, NO₂, CH₃, CF₃, CCl₃, CH₂CH₂F, CH₂CF₃, OH, OCH₃, SH, SCH₃, NH₂, NHCH₃, N(CH₃)₂ and C(O)CH₃.

In an embodiment, R¹⁷ and R¹⁹ are independently selected from H and C₁₋₄alkyl, and alkyl is optionally fluorosubstituted. In an embodiment, R¹⁷ and R¹⁹ are independently selected from H, CH₃, and CF₃. In an embodiment, R¹⁷ and R¹⁹ are both H.

In an embodiment, R¹⁶ and R¹⁷ are joined together to form =X³, and/or R¹⁸ and R¹⁹ are joined together to form =X⁴. In an embodiment, X³ and X⁴ are both O. In an embodiment, X³ and X⁴ are both S. In an embodiment, one of X³ and X⁴ is O and the other is S. In an embodiment, X³ is S and X⁴ is O. In an embodiment, X³ is O and X⁴ is S.

In an embodiment, L′ is C₁₋₃alkylene, and alkylene is optionally fluorosubstituted. In an embodiment, L′ is C₁₋₂alkylene, and alkylene is optionally fluorosubstituted. In an embodiment, L′ is CH₂CH₂, CH₂, CF₂, or CHF. In an embodiment, L′ is CH₂, CF₂, or CHF. In an embodiment, L′ is CH₂.

In an embodiment, R²⁰ and R²¹ are independently selected from H, halo, CHO, CN, NO₂, OH, SH, NH₂, CO₂H, C(O)NH₂, PO₃H, SO₂H, SO₂NH₂, NHSO₂H, C₁₋₁₀alkyl, C₂₋₁₀alkenyl, C₂₋₁₀alkynyl, C₃₋₁₆cycloalkyl, C₃₋₁₆heterocycloalkyl, C₆₋₁₀aryl, C₅₋₁₀heteroaryl, C₁₋₁₀alkyleneC₃₋₁₆cycloalkyl, C₁₋₁₀alkyleneC₃₋₁₆heterocycloalkyl, C₁₋₁₀alkyleneC₆₋₁₀aryl, C₁₋₁₀alkyleneC₅₋₁₆heteroaryl, OC₁₋₁₀alkyl, SC₁₋₁₀alkyl, NH(C_{1- 10}alkyl), N(C₁₋₁₀alkyl)(C₁₋₁₀alkyl), C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), PO(OC₁₋₁₀alkyl) (OC₁₋₁₀alkyl), SO₂C₁₋₁₀alkyl, SO₂NH(C₁₋₁₀alkyl), SO₂N(C₁₋₁₀alkyl)(C₁₋₁₀alkyl), NC₁₋₁₀alkylSO₂(C₁₋₁₀alkyl), and NHSO₂(C₁₋₁₀alkyl) provided R²⁰ and R²¹ are not both H, and each alkyl, alkenyl, alkynyl, cycloalkyl, heterocycloalkyl, aryl, heteroaryl and alkylene group in R²⁰ and R²¹ is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁰), SOR²⁶ SO₂R²⁶, SO₂NR²⁷R²⁸, and NR²⁷SO₂R²⁶ and each alkyl, alkylene, alkenyl and alkynyl is optionally fluorosubstituted.

In an embodiment, R²⁰ and R²¹ are independently selected from H, halo, CHO, CN, NO₂, OH, SH, NH₂, CO₂H, C(O)NH₂, PO₃H, SO₂H, SO₂NH₂, NHSO₂H, C₁₋₆alkyl, C₃₋₁₆cycloalkyl, C₃₋₁₆heterocycloalkyl, C₆₋₁₆aryl, C₅₋₁₆heteroaryl, C₁₋₁₀alkyleneC₃₋₁₆cycloalkyl, C₁₋₆alkyleneC₃₋₁₆heterocycloalkyl, C₁₋₆alkyleneC₆₋₁₀aryl, C₁₋₆alkyleneC516heteroaryl, OC₁₋₆alkyl, SC₁₋₆alkyl, NH(C₁₋₆alkyl), N(C₁₋₆alkyl)(C₁₋₆alkyl), C(O)C₁₋₆alkyl, CO₂C₁₋₆alkyl, C(O)NHC₁₋₁₀alkyl, C(O)N(C₁₋₁₀alkyl)(C₁₋₁₀alkyl), PO (OC₁₋₁₀alkyl)(OC₁₋₁₀alkyl), SO₂C₁₋₁₀alkyl, SO₂NH(C₁₋₁₀alkyl), SO₂N(C₁₋₁₀alkyl)(C₁₋₁₀alkyl), NC₁₋₁₀alkylSO₂(C₁₋₁₀alkyl), and NHSO₂(C₁₋₁₀alkyl), provided R²⁰ and R²¹ are not both H, and each alkyl, cycloalkyl, heterocycloalkyl, aryl, heteroaryl and alkylene group in R²⁰ and R²¹ is optionally substituted with one or more substituents independently selected from halo, NH₂, NHC(NH)NH₂, C₁₋₁₀alkyl, OR^{26,} SR²⁶, NR²⁷R²⁸, C(O)R²⁶, CO₂R²⁶, CONR²⁷R²⁸, PO(OR²⁹)(OR³⁰), SOR²⁶ SO₂R²⁶, SO₂NR²⁷R²⁸, and NR²⁷SO₂R²⁶, and each alkyl and alkylene, is optionally fluorosubstituted.

In an embodiment, R²⁰ and R²¹ are independently selected from H, halo, CHO, CN, NO₂, OH, SH, NH₂, CO₂H, C(O)NH₂, PO₃H, SO₂H, SO₂NH₂, NHSO₂H, C₁₋₆alkyl, OC₁₋₆alkyl, SC₁₋₆alkyl, NH(C₁₋₆alkyl), N(C₁₋₆alkyl)(C₁₋₆alkyl), C(O)C₁₋₆alkyl, CO₂C₁₋₆alkyl, C(O)NHC₁₋₆alkyl, C(O)N(C₁₋₆alkyl)(C₁₋₆alkyl), PO (OC₁₋₆alkyl)(OC₁₋₆alkyl), SO₂C₁₋₆alkyl, SO₂NH(C₁₋₆alkyl), SO₂N(C₁₋₆alkyl)(C₁₋₆alkyl), NC₁₋₆alkylSO₂(C₁₋₆alkyl), and NHSO₂(C₁₋₆alkyl), provided R²⁰ and R²¹ are not both H, and each alkyl and alkylene group in R²⁰ and R²¹ is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁰), SOR²⁶ SO₂R²⁶, SO₂NR²⁷R²⁸, and NR²⁷SO₂R²⁶, and each alkyl and alkylene is optionally fluorosubstituted. In an embodiment, R²⁰ and R²¹ are independently selected from H, F, Cl, CHO, CN, NO₂, OH, SH, NH₂, CO₂H, C(O)NH₂, PO₃H, SO₂H, SO₂NH₂, NHSO₂H, C₁₋₆alkyl, OC₁₋₆alkyl, SC₁₋₆alkyl, NH(C₁₋₄alkyl), N(C₁₋₄alkyl)(C₁₋₄alkyl), C(O)C₁₋₄alkyl, CO₂C₁₋₄alkyl, C(O)NHC₁₋₄alkyl, C(O)N(C₁₋₄alkyl)(C₁₋₄alkyl), PO(OC₁₋₄alkyl)(OC₁₋₄alkyl), SO₂C₁₋₄alkyl, SO₂NH(C₁₋₄alkyl), SO₂N(C₁₋₄alkyl)(C₁₋₄alkyl), NC₁₋₄alkylSO₂(C₁₋₄alkyl), and NHSO₂(C₁₋₄alkyl), provided R²⁰ and R²¹ are not both H, and each alkyl and alkylene group in R²⁰ and R²¹ is optionally substituted with one or more substituents independently selected from F, Cl, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁰), SOR²⁶ SO₂R²⁶, SO₂NR²⁷R²⁸, and NR²⁷SO₂R²⁶ and each alkyl and alkylene is optionally fluorosubstituted.

In an embodiment, one of R²⁰ and R²¹ is selected from CO₂H, C₁₋₆alkyl, OC₁₋₆alkyl, SC₁₋₄alkyl, NH(C₁₋₄alkyl), N(C₁₋₄alkyl)(C₁₋₄alkyl), C(O)C₁₋₄alkyl, and CO₂C₁₋₄alkyl and the other is selected from H, F, Cl, CHO, CN, NO₂, OH, SH, NH₂, CO₂H, C(O)NH_{2,} PO₃H, SO₂H, SO₂NH₂, NHSO₂H, C₁₋₆alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), SO₂N(C₁₋₄alkyl)(C₁₋₄alkyl), NC₁₋₄alkylSO₂(C₁₋₄alkyl), and NHSO₂(C₁₋₄alkyl), and each alkyl group in R²⁰ and R²¹ is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁰), SOR²⁶ SO₂R²⁶, SO₂NR²⁷R²⁸, and NR²⁷SO₂R²⁶, and each alkyl is optionally fluorosubstituted. In an embodiment, one of R²⁰ and R²¹ is selected from CO₂H, C₁₋₆alkyl, OC₁₋₆alkyl, and SC₁₋₄alkyl and the other is selected from H, CO₂H, C(O)NH₂, PO₃H, SO₂H, SO₂NH₂, NHSO₂H, C₁₋₆alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), and SO₂N(C₁₋₄alkyl)(C₁₋₄alkyl) and each alkyl group in R²⁰ and R²¹ is optionally substituted with one or more substituents independently selected from halo, NH₂, NHC(NH)NH₂, C₁₋₆alkyl, OR²⁶, SR²⁶, NR²⁷R²⁸ and CO₂R²⁶, and each alkyl is optionally fluorosubstituted.

In an embodiment, one of R²⁰ and R²¹ is selected from CO₂H and C₁₋₆alkyl optionally substituted with one or more substituents independently selected from NH₂, NHC(NH)NH₂, OR²⁶, SR²⁶, NR²⁷R²⁸ and CO₂R²⁶, and the other is selected from H, CO₂H and C₁₋₆alkyl optionally substituted with one or more OR²⁶ and CO₂R²⁶, and each alkyl is optionally fluorosubstituted. In an embodiment, one of R²⁰ and R²¹ is selected from CO₂H and C₁₋₆alkyl optionally substituted with one or more OR²⁶, NH₂ and CO₂R²⁶, and the other is selected from H, CO₂H and C₁₋₆alkyl optionally substituted with one or more OR²⁶ and each alkyl is optionally fluorosubstituted.

In an embodiment, one of R²⁰ and R²¹ is C₁₋₄alkyl optionally substituted with one OR²⁶ and the other is CO₂H, and the alkyl is optionally fluorosubstituted. In an embodiment, one of R²⁰ and R²¹ is C₁₋₄alkyl and the other is CO₂H, and the alkyl is optionally fluorosubstituted. In an embodiment, one of R²⁰ and R²¹ is CH(CH₃)₂, CH₂CH(CH₃)₂ or CH(CH₃)CH₂CH₃ and the other is CO₂H, and the CH(CH₃)₂, CH₂CH(CH₃)₂ or CH(CH₃)CH₂CH₃ is optionally fluorosubstituted. In an embodiment, one of R²⁰ and R²¹ is C₁₋₄alkyl substituted with one OR²⁶ and the other is CO₂H, and the alkyl is optionally fluorosubstituted. In an embodiment, one of R²⁰ and R²¹ is CH(OH)CH₃ or CH₂OH and the other is CO₂H, and the CH(OH)CH₃ or CH2OH is optionally fluorosubstituted. In an embodiment, one of R²⁰ and R²¹ is CH(OH)CH₃ and the other is CO₂H. In an embodiment, one of R²⁰ and R²¹ is C₁₋₄alkyl and the other is C₁₋₄alkyl optionally substituted with one or more OR²⁶. In an embodiment, R²⁰ and R²¹ are both C₁₋₄alkyl substituted with one or more OR²⁶ and the alkyl is optionally fluorosubstituted. In an embodiment, R²⁰ and R²¹ are both CH₂OH.

In an embodiment, one of R²⁰ and R²¹ is C₁₋₄alkyl optionally substituted with one or more NH₂ and the other is CO₂H or C₁₋₄alkyl optionally substituted with one or more OR²⁶ and each alkyl is optionally fluorosubstituted. In an embodiment, one of R²⁰ and R²¹ is C₁₋₄alkyl optionally substituted with one or more NH₂ and the other is CO₂H and each alkyl is optionally fluorosubstituted. In an embodiment, one of R²⁰ and R²¹ is CH₂CH₂CH₂CH₂NH₂ and the other is CO₂H and the CH₂CH₂CH₂CH₂NH₂ is optionally fluorosubstituted.

In an embodiment, one of R²⁰ and R²¹ is CO₂H or C₁₋₆alkyl optionally substituted with one or more OR²⁶ and CO₂R²⁶, and the other is H and each alkyl is optionally fluorosubstituted. In an embodiment, one of R²⁰ and R²¹ is C₁₋₄alkyl optionally substituted with one or more OR²⁶ and CO₂R²⁶, and the other is H and each alkyl is optionally fluorosubstituted. In an embodiment, one of R²⁰ and R²¹ is CH₂CH₂CH₂OH or CH₂CH₂COOH, and the other is H. In an embodiment, one of R²⁰ and R²¹ is CO₂H and the other is H. In an embodiment, one of R²⁰ and R²¹ is C₁₋₆alkyl and the other is H and the alkyl is optionally fluorosubstituted. In an embodiment, one of R²⁰ and R²¹ is C₁₋₆alkyl and the other is H and the alkyl is optionally fluorosubstituted. In an embodiment, one of R²⁰ and R²¹ is CH₂CH(CH₃)₂ and the other is H. In an embodiment, one of R²⁰ and R²¹ is CO₂H and the other is H.

In an embodiment, one of R²⁰ and R²¹ is selected from C₃₋₁₆cycloalkyl, C₃₋₁₆heterocycloalkyl, C₆₋₁₆aryl, C₅₋₁₆heteroaryl, C₁₋₆alkyleneC₃₋₁₆cycloalkyl, C₁₋₆alkyleneC₃₋₁₆heterocycloalkyl, C₁₋₆alkyleneC₆₋₁₀aryl, and C₁₋₆alkyleneC₅₋₁₆heteroaryl, and the other is selected from F, Cl, CHO, CN, NO₂, OH, SH, NH₂, CO₂H, C(O)NH₂, PO₃H, SO₂H, SO₂NH₂, NHSO₂H, C₁₋₆alkyl, CO₂C₁₋₄alkyl, C(O)NHC₁₋₄alkyl, C(O)N(C₁₋₄alkyl)(C₁₋₄alkyl), PO(OC₁₋₄alkyl) (OC₁₋₄alkyl), SO₂C₁₋₄alkyl, SO₂NH(C₁₋₄alkyl), SO₂N(C₁₋₄alkyl)(C₁₋₄alkyl), NC₁₋₄alkylSO₂(C₁₋₄alkyl), and NHSO₂(C₁₋₄alkyl), and each alkyl, cycloalkyl, heterocycloalkyl, aryl, heteroaryl and alkylene group in R²⁰ and R²¹ is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁰), SOR²⁶ SO₂R²⁶, SO₂NR²⁷R²⁸, and NR²⁷SO₂R²⁸, and each alkyl and alkylene is optionally fluorosubstituted. In an embodiment, one of R²⁰ and R²¹ is selected from C₃₋₁₀cycloalkyl, C₃₋₁₀heterocycloalkyl, C₆₋₁₀aryl, C₅₋₁₀heteroaryl, C₁₋₆alkyleneC₃₋₁₀cycloalkyl, C₁₋₆alkyleneC₃₋₁₀heterocycloalkyl, C₁₋₆alkyleneC₆₋₁₀aryl, and C₁₋₆alkyleneC₅₋₁₀heteroaryl, and the other is selected from CO₂H, C(O)NH₂, PO₃H, SO₂H, SO₂NH₂, NHSO₂H, C₁₋₆alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), and SO₂N(C₁₋₄alkyl)(C₁₋₄alkyl) and each alkyl, cycloalkyl, heterocycloalkyl, aryl, heteroaryl and alkylene group in R²⁰ and R²¹ is optionally substituted with one or more substituents independently selected from F, Cl, NH₂, C₁₋₆alkyl, OR²⁶, SR²⁶, and NR²⁷R²⁸, and each alkyl and alkylene is optionally fluorosubstituted. In an embodiment, one of R²⁰ and R²¹ is selected from C₃₋₁₀cycloalkyl, C₃₋₁₀heterocycloalkyl, C₆₋₁₀aryl, C₅₋₁₀heteroaryl, C₁₋₄alkyleneC₃₋₁₀cycloalkyl, C₁₋₄alkyleneC₃₋₁₀heterocycloalkyl, C₁₋₄alkyleneC₆₋₁₀aryl, and C₁₋₄alkyleneC₅₋₁₀heteroaryl, and each cycloalkyl, heterocycloalkyl, aryl, heteroaryl and each alkylene group is optionally substituted with one or more substituents independently selected from F, Cl, NH₂, C₁₋₆alkyl, OR²⁶, SR²⁶, and NR²⁷R²⁸, and the other is selected from CO₂H, C(O)NH₂, PO₃H, SO₂H, SO₂NH₂, NHSO₂H, C₁₋₆alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), and SO₂N(C₁₋₄alkyl)(C₁₋₄alkyl) and each alkyl group is optionally substituted with one or more substituents independently selected from F, Cl, NH₂, C₁₋₆alkyl, OR²⁶, SR²⁶, and NR²⁷R²⁸ and each alkyl and alkylene is optionally fluorosubstituted. In an embodiment, one of R²⁰ and R²¹ is selected from C₁₋₄alkyleneC₃₋₁₀cycloalkyl, C₁₋₄alkyleneC₃₋₁₀heterocycloalkyl, C₁₋₄alkyleneC₆₋₁₀aryl, and C₁₋₄alkyleneC₅₋₁₀heteroaryl, and each aryl, heteroaryl and each alkylene group is optionally substituted with one or more substituents independently selected from F, Cl, NH₂, C₁₋₆alkyl, OR²⁶, SR²⁶, and NR²⁷R²⁸, and the other is selected from CO₂H, C(O)NH₂, PO₃H, SO₂H, SO₂NH₂, NHSO2H, C₁₋₆alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), and SO₂N(C₁₋₄alkyl)(C₁₋₄alkyl) and each alkyl group is optionally substituted with one or more substituents independently selected from F, Cl, NH₂, C₋₆alkyl, OR²⁶, SR²⁶, and NR²⁷R²⁸, and each alkyl and alkylene is optionally fluorosubstituted.

In an embodiment, the aryl in R²⁰ and R²¹ is selected from phenyl, naphthyl and indanyl. In an embodiment the aryl in R²⁰ and R²¹ is phenyl.

In an embodiment, the heteroaryl in R²⁰ and R²¹ is selected from pyrrolyl, imidazolyl, oxazolyl, pyrazolyl, pyridinyl, pyrimidinyl, thiophenyl, indolyl, isoindolyl, quinolinyl, and isoquinolinyl.

In an embodiment, cycloalkyl in R²⁰ and R²¹ is selected from cyclopropyl, cyclobutyl, cyclopentyl and cyclohexyl.

In an embodiment, the heterocycloalkyl in R²⁰ and R²¹ is selected from azetidinyl, oxetanyl, tetrohydrofuranyl, tetrahydrothiophenyl, pyrrolidinyl, imidazolidinyl, pyrazolidinyl, thiazolidinyl, isothiazolidinyl, dioxolanyl, dithiolanyl, thianyl, piperidinyl, piperazinyl, tetrahydropyranyl, thiomorpholinyl, morpholinyl, and dioxanyl.

In an embodiment, R²⁰ and R²¹ together with the carbon to which they are attached form C₃₋₈cycloalkyl and the cycloalkyl is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁰), SOR²⁶ SO₂R²⁶, SO₂NR²⁷R²⁸, and NR²⁷SO₂R²⁶, and each alkyl is optionally fluorosubstituted. In an embodiment, R²⁰ and R²¹ together with the carbon to which they are attached form C₄₋₆cycloalkyl and the cycloalkyl is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁰), SOR²⁶ SO₂R²⁶, SO₂NR²⁷R²⁸, and NR²⁷SO₂R²⁶, and each alkyl is optionally fluorosubstituted. In an embodiment, R²⁰ and R²¹ together with the carbon to which they are attached form C₄₋₆cycloalkyl. In an embodiment, R²⁰ and R²¹ together with the carbon to which they are attached form cyclobutyl, cyclopentyl, cyclohexyl or cycloheptyl. In an embodiment, R²⁰ and R²¹ together with the carbon to which they are attached form cyclohexyl.

In an embodiment, R²² is selected from H, C₁₋₆alkyl and C₁₋₆alkyl substituted with one or more substituents selected from halo, NH₂, OH, OC₁₋₆alkyl, NH(C₁₋₆alkyl), N(C₁₋₆alkyl)₂, CO₂H, C(O)₂C₁₋₆alkyl, CONC₁₋₆alkyl, CONH₂, CON(C₁₋₆alkyl)₂, and each alkyl is optionally fluorosubstituted. In an embodiment, R²² is selected from H, C₁₋₆alkyl and C₁₋₆alkyl optionally substituted with one or more substituents selected from halo, NH₂, OH, CO₂H and CONH₂ and alkyl is optionally fluorosubstituted. In an embodiment, R²² is selected from H, C₁₋₆alkyl and C₁₋₆alkyl substituted with one or more substituents selected from OH and CO₂H and each alkyl is optionally fluorosubstituted. In an embodiment, R²² is C₁₋₆alkyl substituted with one or more OH and alkyl is optionally fluorosubstituted. In an embodiment, R²² is CH₂CH₂OH. In an embodiment, R²² is selected from H or C₁₋₆alkyl, and alkyl is optionally fluorosubstituted. In an embodiment, R²² is selected from H, CH₃, CHF₂, or CF₃. In an embodiment, R²² is selected from H and CH₃. In an embodiment, R²² is CH₃ In an embodiment, R²² is H.

In an embodiment, R^{22a} is selected from H, C₁₋₆alkyl and C₁₋₆alkyl substituted with one or more substituents selected from halo, NH₂, OH, OC₁₋₆alkyl, NH(C₁₋₆alkyl), N(C₁₋₆alkyl)₂, CO₂H, C(O)₂C₁₋₆alkyl, CONC₁₋₆alkyl, CONH₂, CON(C₁₋₆alkyl)₂ and alkyl is optionally fluorosubstituted. In an embodiment, R^{22a} is selected from H, C₁₋₆alkyl and C₁₋₆alkyl substituted with one or more substituents selected from halo, NH₂, OH, CO₂H and CONH₂ and each alkyl is optionally fluorosubstituted. In an embodiment, R^{22a} is selected from H, C₁₋₆alkyl and C₁₋₆alkyl substituted with one or more substituents selected from OH and CO₂H and each alkyl is optionally fluorosubstituted. In an embodiment, R^{22a} is C₁₋₆alkyl substituted with one or more CO₂H and alkyl is optionally fluorosubstituted. In an embodiment, R^{22a} is CH₂CO₂H. In an embodiment, R^{22a} is selected from H or C₁₋₆alkyl, and alkyl is optionally fluorosubstituted. In an embodiment, R^{22a} is CH₂CH₂C(CH₃)₂. In an embodiment, R^{22a} is selected from H, CH₃, CHF₂, or CF₃. In an embodiment, R^{22a} is selected from H and CH3.ln an embodiment, R^{22a} is CH₃. In an embodiment, R^{22a} is H.

In an embodiment, one of R²⁰ and R²¹ is selected from CO₂H and C₁₋₆alkyl optionally substituted with one or more OR²⁶, NH₂ and CO₂ R²⁶, and the other is selected from H, CO₂H and C₁₋₆alkyl optionally substituted with one or more OR²⁶, R²² is selected from H, C₁₋₆alkyl and C₁₋₆alkyl substituted with one or more substituents selected from OH and CO₂H and R^{22a} is selected from H, C₁₋₆alkyl and C₁₋₆alkyl substituted with one or more substituents selected from OH and CO₂H, and each alkyl is optionally fluorosubstituted.

In an embodiment, one of R²⁰ and R²¹ is C₁₋₄alkyl optionally substituted with one OR²⁶ and the other is CO₂H, R²² is selected from H, C₁₋₆alkyl and R^{22a} is H and each alkyl is optionally fluorosubstituted. In an embodiment, one of R²⁰ and R²¹ is C₁₋₄alkyl and the other is CO₂H, R²² is H and R^{22a} is H and the alkyl is optionally fluorosubstituted. In an embodiment, one of R²⁰ and R²¹ is CH(CH₃)₂, CH₂CH(CH₃)₂ or CH(CH₃)CH₂CH₃ and the other is CO₂H, R²² is H and R^{22a} is H and the CH(CH₃)₂, CH₂CH(CH₃)₂ or CH(CH₃)CH₂CH₃ is optionally fluorosubstituted. In an embodiment, one of R²⁰ and R²¹ is C₁₋₄alkyl substituted with one OR²⁶ and the other is CO₂H, R²² is H and R^{22a} is H and the alkyl is optionally fluorosubstituted. In an embodiment, one of R²⁰ and R²¹ CH(OH)CH₃ or CH₂OH and the other is CO₂H, R²² is H and R^{22a} is H and the CH(OH)CH₃ or CH₂OH is optionally fluorosubstituted. In an embodiment, one of R²⁰ and R²¹ is CH(OH)CH₃ and the other is CO₂H, R²² is H and R^{22a} is H. In an embodiment, R²⁰ and R²¹ are both C₁₋₄alkyl substituted with one or more OR²⁶, R²² is selected from H and CH₃ and R^{22a} is H and each alkyl is optionally fluorosubstituted. In an embodiment, R²⁰ and R²¹ are both CH₂OH, R²² is selected from H and CH₃ and R^{22a} is H.

In an embodiment, one of R²⁰ and R²¹ is C₁₋₄alkyl optionally substituted with one or more NH₂ and the other is CO₂H or C₁₋₄alkyl optionally substituted with one or more OR²⁶, R²² is selected from H and CH₃ and R^{22a} is H and each alkyl is optionally fluorosubstituted. In an embodiment, one of R²⁰ and R²¹ is C₁₋₄alkyl optionally substituted with one or more NH₂ and the other is CO₂H, R²² is selected from H and CH₃ and R^{22a} is H and each alkyl is optionally fluorosubstituted. In an embodiment, one of R²⁰ and R²¹ is CH₂CH₂CH₂CH₂NH₂ and the other is CO₂H, R²² is selected from H and CH₃ and R^{22a} is H.

In an embodiment, one of R²⁰ and R²¹ is C₁₋₆alkyl optionally substituted with one or more OR²⁶ and COOH, and the other is H, R²² is H, and R^{22a} is H, and each alkyl is optionally fluorosubstituted. In an embodiment, one of R²⁰ and R²¹ is C₁₋₄alkyl optionally substituted with one or more OR²⁶ and COOH, and the other is H, R²² is H, and R^{22a} is H. In an embodiment, one of R²⁰ and R²¹ is CH₂CH₂CH₂OH or CH₂CH₂COOH, and the other is H, R²² is H, and R^{22a} is H.

In an embodiment, one of R²⁰ and R²¹ is C₁₋₆alkyl and the other is H, R²² is H and R^{22a} is C₁₋₆alkyl and each alkyl is optionally fluorosubstituted.

In an embodiment, one of R²⁰ and R²¹ is CO₂H and the other is H, R²² is H and R^{22a} is C₁₋₆alkyl substituted with one or more substituents selected from OH and CO₂H and each alkyl is optionally fluorosubstituted. In an embodiment, one of R²⁰ and R²¹ is CO₂H and the other is H, R²² is H and R^{22a} is CH₂CO₂H. In an embodiment, one of R²⁰ and R²¹ is CO₂H and the other is H, R²² is H and R^{22a} is C₁₋₄alkyl substituted with one or more substituents selected from OH and CO₂H and each alkyl is optionally fluorosubstituted. In an embodiment, R²⁰ and R²¹ together with the carbon to which they are attached form cyclohexyl, R²² is selected from H and C₁₋₆alkyl substituted with one or more OH and R^{22a} is H and each alkyl is optionally fluorosubstituted. In an embodiment, R²⁰ and R²¹ together with the carbon to which they are attached form cyclohexyl, R²² is selected from H and CH₂CH₂OH C₁, and R^{22a} is H.

In an embodiment, R²⁶, R²⁷, R²⁸, R²⁹, and R³⁰ are independently selected from H and C₁₋₄alkyl; and alkyl is optionally fluorosubstituted. In an embodiment, R²⁶, R²⁷, R²⁸, R²⁹, and R³⁰ are independently selected from H and CH₃, and CH₃ is optionally fluorosubstituted.

In an embodiment, the compounds of Formula II are selected from compounds of the following Formulae:

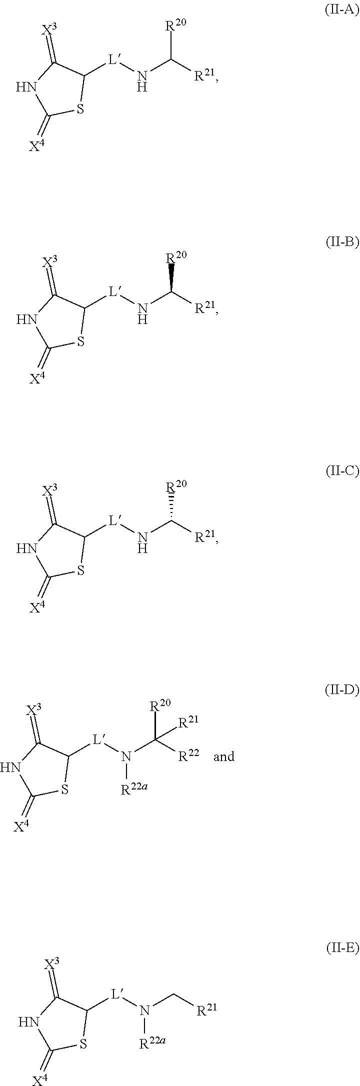

or a pharmaceutically acceptable salt and/or solvate thereof.

In an embodiment, the compound of Formula II is selected from the compounds listed below:

| Compound I.D | Chemical Structure |
|---|---|
| II-1 | 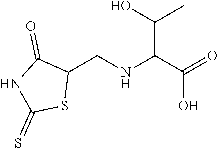 [SMILES: CC(O)C(NCC1SC(=S)NC1=O)C(=O)O] |
| II-2 | 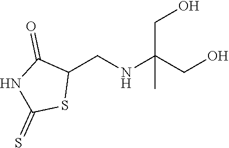 |
| II-3 | 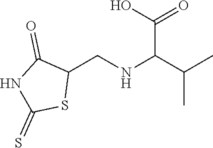 [SMILES: CC(C)C(NCC1SC(=S)NC1=O)C(=O)O] |
| II-4 | 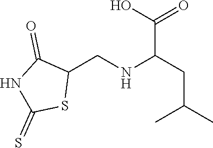 [SMILES: CC(C)CC(NCC1SC(=S)NC1=O)C(=O)O] |
| II-5 | 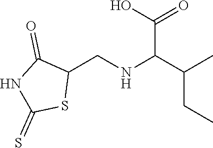 [SMILES: CCC(C)C(NCC1SC(=S)NC1=O)C(=O)O] |
| II-6 | 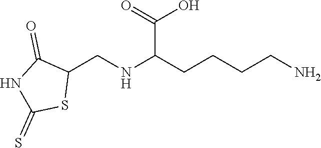 [SMILES: NCCCCC(NCC1SC(=S)NC1=O)C(=O)O] |
| II-7 | 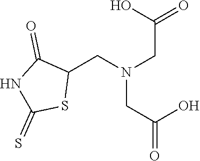 [SMILES: O=C(O)CN(CC(=O)O)CC1SC(=S)NC1=O] |
| II-8 | 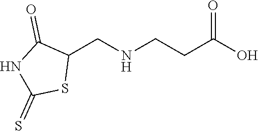 [SMILES: O=C(O)CCNCC1SC(=S)NC1=O] |
| II-9 | 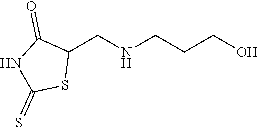 [SMILES: O=C1NC(=S)SC1CNCCCO] |
| II-10 | 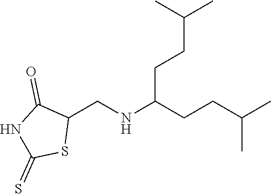 [SMILES: CC(C)CCC(CCC(C)C)NCC1SC(=S)NC1=O] |
| II-11 | 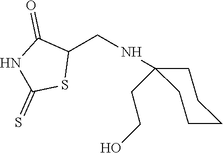 [SMILES: O=C1NC(=S)SC1CNC1(CCO)CCCCC1] |
| II-12 | 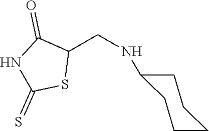 [SMILES: O=C1NC(=S)SC1CNC1CCCCC1] |

or a pharmaceutically acceptable salt and/or solvate thereof.

In an embodiment, the compound of Formula II is selected from the compounds listed below:

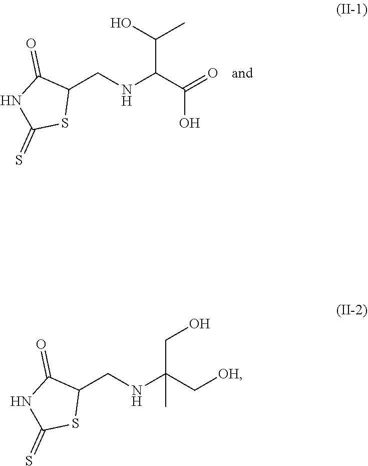 [SMILES: CC(O)C(NCC1SC(=S)NC1=O)C(=O)O ; CC(CO)(CO)NCC1SC(=S)NC1=O]

or a pharmaceutically acceptable salt and/or solvate thereof.

The application also includes a use of one or more compounds of Formula II or a pharmaceutically acceptable salt and/or solvate thereof for inhibiting interactions between Cav3.2 and USP5 in a cell as well as a use of one or more compounds or compositions of the application for the preparation of a medicament for inhibiting interactions between Cav3.2 and USP5 in a cell. The application further includes one or more compounds of Formula II or a pharmaceutically acceptable salt and/or solvate thereof for use in inhibiting interactions between Cav3.2 and USP5.

As the compounds of Formula II have been shown to be capable of inhibiting interactions between Cav3.2 and USP5, one or more compounds of Formula II are useful for treating diseases, disorders or conditions by inhibiting interactions between Cav3.2 and USP5. Therefore, the compounds of Formula II are useful as medicaments. Accordingly, the present application includes compounds of Formula II for use as a medicament.

The present application also includes a method of treating a disease, disorder or condition that is treatable by inhibiting interactions between Cav3.2 and USP5 comprising administering a therapeutically effective amount of one or more compounds of Formula II or a pharmaceutically acceptable salt and/or solvate thereof to a subject in need thereof.

The application also includes a use of one or more compounds Formula II or a pharmaceutically acceptable salt and/or solvate thereof for treating a disease, disorder or condition that is treatable by inhibiting interactions between Cav3.2 and USP5 in a cell as well as a use of one or more compounds of Formula II or a pharmaceutically acceptable salt and/or solvate thereof for preparing a medicament for treating a disease, disorder or condition that is treatable by inhibiting interactions between Cav3.2 and USP5 in a cell. The application further includes one or more compounds of Formula II or a pharmaceutically acceptable salt and/or solvate thereof for use in treating a disease, disorder or condition that is treatable by inhibiting interactions between Cav3.2 and USP5.

The present application also includes a method for inhibiting Cav3.2 T-type calcium channel (Cav3.2) function and/or expression by inhibiting interactions between Cav3.2 and ubiquitin specific peptidase 5 (USP5) in a cell, either in a biological sample or in a patient, comprising administering an effective amount of one or more compounds of Formula II or a pharmaceutically acceptable salt and/or solvate thereof to the cell.

The application also includes a use of one or more compounds of Formula II or a pharmaceutically acceptable salt and/or solvate thereof for inhibiting Cav3.2 function and/or expression by inhibiting interactions between Cav3.2 and USP5 in a cell as well as a use of one or more compounds of Formula II or a pharmaceutically acceptable salt and/or solvate thereof for the preparation of a medicament for inhibiting Cav3.2 function and/or expression by inhibiting interactions between Cav3.2 and USP5 in a cell. The application further includes one or more compounds of Formula II or a pharmaceutically acceptable salt and/or solvate thereof for use in inhibiting Cav3.2 function and/or expression by inhibiting interactions between Cav3.2 and USP5 in a cell.

The application further includes one or more compounds of Formula II or a pharmaceutically acceptable salt and/or solvate thereof for use in inhibiting Cav3.2 function and/or expression by inhibiting interactions between Cav3.2 and USPS.The application also includes a method of treating a disease, disorder or condition mediated or treatable by inhibiting Cav3.2 function and/or expression by inhibiting interactions between Cav3.2 and USP5 in a cell comprising administering a therapeutically effective amount of one or more compounds of Formula II or a pharmaceutically acceptable salt and/or solvate to a subject in need thereof.

The application also includes a use of one or more compounds of Formula II or a pharmaceutically acceptable salt and/or solvate thereof for treating a disease, disorder or condition that is treatable by inhibiting Cav3.2 function and/or expression by inhibiting interactions between Cav3.2 and USP5 in a cell as well as a use of one or more compounds of Formula II or a pharmaceutically acceptable salt and/or solvate thereof for the preparation of a medicament for treating a disease, disorder or condition that is treatable by inhibiting Cav3.2 function and/or expression by inhibiting interactions between Cav3.2 and USP5 in a cell. The application further includes one or more compounds of Formula II or a pharmaceutically acceptable salt and/or solvate thereof for use in treating a disease, disorder or condition that is treatable by inhibiting Cav3.2 function and/or expression by inhibiting interactions between Cav3.2 and USP5.

As USP5 is a deubiquitinizing enzyme, and the compounds of Formula II have been shown to inhibit interactions between Cav3.2 and USP5, the compounds of Formula II may be useful for inhibiting Cav3.2 deubiquitination. Accordingly, the present application also includes a method for inhibiting Cav3.2 deubiquitination in a cell, either in a biological sample or in a patient, comprising administering an effective amount of one or more compounds of Formula II or a pharmaceutically acceptable salt and/or solvate thereof to the cell.

The application also includes a use of one or more compounds of Formula II or a pharmaceutically acceptable salt and/or solvate thereof for inhibiting Cav3.2 deubiquitination in a cell as well as a use of one or more compounds of Formula II or a pharmaceutically acceptable salt and/or solvate thereof for the preparation of a medicament for inhibiting Cav3.2 deubiquitination in a cell. The application further includes one or more compounds of Formula II or a pharmaceutically acceptable salt and/or solvate thereof for use in inhibiting Cav3.2 deubiquitination.

As the compounds of Formula II have been shown to be capable of inhibiting interactions between Cav3.2 and the deubiquitinase USP5, therefore the one or more compounds of Formula II are useful for treating diseases, disorders or conditions by inhibiting Cav3.2 deubiquitination. Therefore, the present application also includes a method of treating a disease, disorder or condition that is treatable by inhibiting Cav3.2 deubiquitination comprising administering a therapeutically effective amount of one or more compounds of Formula II or a pharmaceutically acceptable salt and/or solvate thereof to a subject in need thereof.

The application also includes a use of one or more compounds of Formula II or a pharmaceutically acceptable salt and/or solvate thereof for treating a disease, disorder or condition that is treatable by inhibiting Cav3.2 deubiquitination in a cell as well as a use of one or more compounds of Formula II or a pharmaceutically acceptable salt and/or solvate thereof for the preparation of a medicament for treating a disease, disorder or condition that is treatable by inhibiting Cav3.2 deubiquitination in a cell. The application further includes one or more compounds of Formula II or a pharmaceutically acceptable salt and/or solvate thereof for use in treating a disease, disorder or condition that is treatable by inhibiting Cav3.2 deubiquitination.

In an embodiment, the disease, disorder or condition treatable by inhibiting interactions between Cav3.2 and USP5 or inhibiting Cav3.2 deubiquitination is pain. Accordingly, the present application also includes a method of treating pain comprising administering a therapeutically effective amount of one or more compounds of Formula II or a pharmaceutically acceptable salt and/or solvate thereof to a subject in need thereof. The present application also includes a use of one or more compounds of Formula II or a pharmaceutically acceptable salt and/or solvate thereof for treatment of pain as well as a use of one or more compounds of Formula II or a pharmaceutically acceptable salt and/or solvate thereof for the preparation of a medicament to treat pain. The application further includes one or more compounds of Formula II or a pharmaceutically acceptable salt and/or solvate thereof for use in pain. In an embodiment, the treatment is in an amount effective to ameliorate at least one symptom of pain, for example, reduced sensation and duration, among others, in a subject in need of such treatment.

In an embodiment, the one or more compounds of Formula II are useful as an analgesic in the treatment of pain.

In an embodiment, the pain is acute or chronic pain.

In an embodiment, the chronic pain is any persistent or recurrent pain lasting longer than about 3 months. In an embodiment, the chronic pain is any persistent or recurrent pain lasting longer than about 6 months. In an embodiment, the chronic pain is selected from chronic primary pain, chronic cancer pain, chronic postsurgical and posttraumatic pain, chronic neuropathic pain, orofacial pain, chronic visceral pain, chronic musculoskeletal pain. In an embodiment, the chronic pain is selected from chronic primary pain, chronic neuropathic pain, chronic headache and orofacial pain, chronic visceral pain, and chronic musculoskeletal pain. In an embodiment, the chronic pain is selected from chronic primary pain, chronic neuropathic pain and chronic visceral pain. In an embodiment, the chronic pain is selected from chronic neuropathic pain and chronic visceral pain.

In an embodiment, the pain is nociceptive pain, inflammatory pain, or neuropathic pain. In an embodiment, the pain is inflammatory pain or neuropathic pain. In an embodiment, the pain is inflammatory pain. In an embodiment, the pain is neuropathic pain.

In an embodiment, the neuropathic pain is chronic neuropathic pain. In an embodiment, the chronic neuropathic pain is central pain syndrome, complex regional pain syndrome, diabetic peripheral neuropathic pain, shingles, postherpetic neuralgia, or trigeminal neuralgia.

In an embodiment, the central pain syndrome is pain that is associated with diseases, disorders or conditions associated with damage to the central nervous system. In an embodiment, the diseases, disorders or conditions associated with damage to the central nervous system are selected from one or more of multiple sclerosis, and tumors.

In an embodiment, the diabetic peripheral neuropathic pain is associated with diabetes. In an embodiment, the diabetic peripheral neuropathic pain is associated with nerve damage in the feet, legs, hands, or arms associated with diabetes. Therefore, in an embodiment, the pain is pain is associated with diabetes.

In an embodiment, the inflammatory pain is pain associated with diseases, disorders or conditions associated with inflammation. In an embodiment, the diseases, disorders or conditions associated with inflammation are selected from one or more of diabetes, cardiovascular disease (CVD), arthritis, chronic obstructive pulmonary disease (COPD), asthma, bronchitis, menstrual cramps, tendinitis, bursitis, skin-related conditions (such as psoriasis, eczema, burns and dermatitis), and post-operative inflammation. In an embodiment, the diseases, disorders or conditions associated with inflammation is diabetes.

In an embodiment, the arthritis is, but is not limited to, rheumatoid arthritis, spondyloarthropathies, gouty arthritis, osteoarthritis, systemic lupus erythematosus or juvenile arthritis.

In an embodiment, the diseases, disorders or conditions associated with inflammation are selected from one or more of inflammatory bowel disease, Crohn's disease, gastritis, irritable bowel syndrome and ulcerative colitis.

In an embodiment, the diseases, disorders or conditions associated with inflammation are selected from one or more of vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, and the like.

In an embodiment, the diseases, disorders or conditions associated with inflammation are ophthalmic diseases, including but not limited to retinitis, retinopathies, uveitis, ocular photophobia, and acute injury to the eye tissue.

In an embodiment, the disease, disorder or condition associated with inflammation is pulmonary inflammation, including but not limited to, that associated with viral infections and cystic fibrosis.

In an embodiment, the disease, disorder or condition associated with inflammation is a central nervous system disorder such as cortical dementias including Alzheimer's disease.

In an embodiment, the disease, disorder or condition associated with inflammation is selected from allergic rhinitis, respiratory distress syndrome, endotoxin shock syndrome, atherosclerosis and central nervous system damage resulting from stroke, ischemia and trauma.

In further embodiments, the present application also includes a method of treating a disease, disorder or condition mediated or treatable by inhibiting interactions between Cav3.2 and USPS or inhibiting Cav3.2 deubiquitination comprising administering a therapeutically effective amount of one or more compounds of Formula II in combination with another known agent useful for treatment of a disease, disorder or condition mediated or treatable by condition treatable by inhibiting interactions between Cav3.2 and USPS or inhibiting Cav3.2 deubiquitination to a subject in need thereof. The present application also includes a use of one or more compounds of Formula II in combination with a known agent useful for treatment of a disease, disorder or condition mediated or treatable by inhibiting interactions between Cav3.2 and USPS or inhibiting Cav3.2 deubiquitination protein, and one or more compounds of Formula II for treatment of a disease, disorder or condition mediated or treatable by inhibiting interactions between Cav3.2 and USPS or inhibiting Cav3.2 deubiquitination.

In a further embodiment, the disease, disorder or condition mediated or treatable by inhibiting interactions between Cav3.2 and USPS or inhibiting Cav3.2 deubiquitination protein is pain and the one or more compounds of Formula II are administered in combination with one or more additional pain treatments.

In an embodiment, effective amounts vary according to factors such as the disease state, age, sex and/or weight of the subject. In a further embodiment, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

In an embodiment, the compounds of Formula II are administered at least once a week. However, in another embodiment, the compounds of Formula II are administered to the subject from about one time per two weeks, three weeks or one month. In another embodiment, the compounds are administered about one time per week to about once daily. In another embodiment, the compounds of Formula II are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds of Formula II are administered to the subject in an amount and for duration sufficient to treat the subject.

In an embodiment, the subject is a mammal. In another embodiment, the subject is human.

Compounds of Formula II are either used alone or in combination with other known agents useful for treating diseases, disorders or conditions that are mediated or treatable by inhibiting interactions between Cav3.2 and USPS or inhibiting Cav3.2 deubiquitination. When used in combination with other agents useful in treating diseases, disorders or conditions mediated or treatable by inhibiting interactions between Cav3.2 and USPS or inhibiting Cav3.2 deubiquitination, it is an embodiment that a compound of Formula II is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In an embodiment, a compound of Formula II is administered with another therapeutic agent simultaneously or sequentially in separate unit dosage forms or together in a single unit dosage form. Accordingly, the present application provides a single unit dosage form comprising one or more compounds of Formula II, an additional therapeutic agent, and a pharmaceutically acceptable carrier.

The dosage of a compound of Formula II varies depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. In some embodiments, a compound of Formula II is administered initially in a suitable dosage that is adjusted as required, depending on the clinical response. Dosages will generally be selected to maintain a serum level of the compound of the application from about 0.01 μg/cc to about 1000 μg/cc, or about 0.1 μg/cc to about 100 μg/cc. As a representative example, oral dosages of one or more compounds of Formula II will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg.

### II. Compounds of the Application

The present application includes a compound of Formula (I), or a pharmaceutically acceptable salt, solvate and/or prodrug thereof:

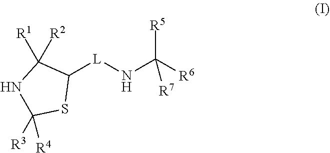

wherein:
R¹ and R³ are independently selected from H, halo, CN, NO₂, C₁₋₆alkyl, C₁₋₆haloalkyl, OR⁸, SR⁸, NR⁹R¹⁰, C(O)R^{8,} CO₂R⁸, CONR⁹R¹⁰, SOR⁸, SO₂R⁸, and SO₂NR⁹R¹⁰;
R² and R⁴ are independently selected from H and C₁₋₆alkyl; or
R¹ and R² are joined together to form =X¹, and/or R³ and R⁴ are joined together to form =X²;
X¹ and X² are independently selected from O and S;

### L is C₁₋₄alkylene;

R⁵ and R⁶ are independently selected from halo, CHO, CN, NO₂, OH, SH, NH₂, CO₂H, C(O)NH₂, POSH, SO₂H, SO₂NH₂, NHSO₂H, C₁₋₁₀alkyl, C₂₋₁₀alkenyl, C₂₋₁₀alkynyl, C₃₋₁₆cycloalkyl, C₃₋₁₆heterocycloalkyl, C₆₋₁₀aryl, C₆₋₁₀heteroaryl, C₁₋₁₀alkyleneC₃₋₁₆cycloalkyl, C₁₋₁₀alkyleneC₃₋₁₆heterocycloalkyl, C₁₋₁₀alkyleneC₆₋₁₀aryl, C₁₋₁₀alkyleneC₆₋₁₆heteroaryl, OC₁₋₁₀alkyl, SC₁₋₁₀alkyl, NH(C₁₋₁₀alkyl), N(C₁₋₁₀alkyl)(C₁₋₁₀alkyl), C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), PO(OC₁₋₁₀alkyl)(OC₁₋₁₀alkyl), SO₂C₁₋₁₀alkyl, SO₂NH(C₁₋₁₀alkyl), SO₂N(C₁₀alkyl)(C₁₋₁₀alkyl), NC₁₋₁₀alkylSO₂(C₁₋₁₀alkyl), and NHSO₂(C₁₋₁₀alkyl),
and each alkyl, alkenyl, alkynyl, cycloalkyl, heterocycloalkyl, aryl, heteroaryl and alkylene group in R⁵ and R⁶ is optionally substituted with one or more substituents independently selected from halo, NH₂, NHC(NH)NH₂, C₁₋₆alkyl, OR¹¹, SR¹¹, NR¹²R¹³, C(O)R¹¹, CO₂R¹¹, CONR¹²R¹³, PO(OR¹⁴)(OR¹⁵), SOR¹¹, SO₂R¹¹, SO₂NR¹²R¹³, and NR¹²SO₂R¹¹;
R⁷ is selected from H and C₁₋₆alkyl; or
R⁸, R⁹, and R¹⁰ are independently selected from H, C₁₋₆alkyl, C₂₋₆alkenyl and C₂₋₆alkynyl;
R¹¹, R¹², R¹³, R¹⁴, and R¹⁵ are independently selected from H and C₁₋₆alkyl; and each alkyl, alkylene, alkenyl and alkynyl is optionally fluorosubstituted,
provided R⁵ is not —(CH(OH)CH₃, —(CH(CH₃)₂, or —(CH₂)₄NH₂ when R⁶ is COOH, R⁷ is H, L is CH₂, R¹ and R² are joined together to form ═O, and R³ and R⁴ are joined together to form ═S; and
R⁵ and R⁶ are not both —CH₂OH when R⁷ is CH₃, L is CH₂, R¹ and R² are joined together to form ═O, and R³ and R⁴ are joined together to form ═S.

In an embodiment, R¹ and R³ are independently selected from H, halo, CN, NO₂, C₁₋₆alkyl, C₁₋₆haloalkyl, OR⁸, SR⁸, NR⁹R¹⁰, C(O)R⁸, CO₂R⁸, CONR⁹R¹⁰, SOR⁸, SO²R⁸, and SO₂NR⁹R¹⁰, and each alkyl, alkylene, alkenyl and alkynyl is optionally fluorosubstituted. In an embodiment, R¹ and R³ are independently selected from H, F, Cl, CN, NO₂, C₁₋₃alkyl, C₁₋₃haloalkyl, OR⁸, SR⁸, NR⁹R¹⁰, C(O)R⁸, CO₂R⁸, CONR⁹R¹⁰, SOR⁸, SO₂R⁸, and SO₂NR⁹R¹⁰, and each alkyl, alkylene, alkenyl and alkynyl is optionally fluorosubstituted. In an embodiment, R¹ and R³ are independently selected from H, F, Cl, CN, NO₂, C₁₋₃alkyl, C₁₋₃haloalkyl, OR⁸, SR⁸, NR⁹R¹⁰, C(O)R⁸, CO₂R⁸, C(O)NR⁹R¹⁰, SOR⁸ , SO₂R⁸, and SO₂NR⁹R¹⁰ and each alkyl, alkylene, alkenyl and alkynyl is optionally fluorosubstituted. In an embodiment, R⁸, R⁹, and R¹⁰ are independently selected from H, C₁₋₄alkyl, C₂₋₆alkynyl, and C₂₋₆alkymyl, and each alkyl, alkylene, alkenyl and alkynyl is optionally fluorosubstituted. In an embodiment, R⁸, R⁹, and R¹⁰ are independently selected from H and C₁₋₄alkyl. In an embodiment, R¹ and R³ are independently selected from H, F, Cl, CN, NO₂, CH₃, CF₃, CCl₃, CH₂CH₂F, CH₂CF₃, OH, OCH₃, SH, SCH₃, NH₂, NHCH₃, N(CH₃)₂, CHO, C(O)CH₃, CO₂H, CO₂CH₃, CONH₂, CON(CH₃)₂, CONHCH₃, SOCH₃, SO₂H, SO₂CH₃, SO₂N(CH₃)₂, SO₂NHCH₃, and SO₂N(CH₃)₂. In an embodiment, R¹ and R³ are independently selected from H, F, Cl, CN, NO₂, CH₃, CF₃, CCl₃, CH₂CH₂F, CH₂CF₃, OH, OCH₃, SH, SCH3, NH₂, NHCH₃, N(CH₃)₂, CHO, and C(O)CH₃. In an embodiment, R¹ and R³ are independently selected from H, F, CN, NO₂, CH₃, CF₃, CCl₃, CH₂CH₂F, CH₂CF₃, OH, OCH₃, SH, SCH₃, NH₂, NHCH₃, N(CH₃)₂ and C(O)CH₃.

In an embodiment, R² and R⁴ are independently selected from H and C₁₋₄alkyl, and alkyl is optionally fluorosubstituted. In an embodiment, R² and R⁴ are independently selected from H, CH₃, and CF₃. In an embodiment, R² and R⁴ are both H.

In an embodiment, R¹ and R² are joined together to form =X¹, and/or R³ and R⁴ are joined together to form =X². In an embodiment, X¹ and X² are both O. In an embodiment, X¹ and X² are both S. In an embodiment, one of X¹ and X² is O and the other is S. In an embodiment, X¹ is S and X² is O. In an embodiment, X¹ is O and X² is S.

In an embodiment, L is C₁₋₃alkylene, and alkylene is optionally fluorosubstituted. In an embodiment, L is C₁₋₂alkylene, and alkylene is optionally fluorosubstituted. In an embodiment, L is CH₂CH₂, CH₂, CF₂, or CHF. In an embodiment, L is CH₂, CF₂, or CHF.

In an embodiment, R⁵ and R⁶ are independently selected from halo, CHO, CN, NO₂, OH, SH, NH₂, CO₂H, C(O)NH₂, PO₃H, SO₂H, SO₂NH₂, NHSO₂H, C₁₋₆alkyl, C₂₋₆alkenyl, C₂₋₆alkynyl, C₃₋₁₆cycloalkyl, C₃₋₁₆heterocycloalkyl, C₆₋₁₆aryl, C₅₋₁₆heteroaryl, C₁₋₆alkyleneC₃₋₁₆cycloalkyl, C₁₋₆alkyleneC₃₋₁₆heterocycloalkyl, C₁₋₆alkyleneC₆₋₁₀aryl, C₁₋₆alkyleneC₅₋₁₆heteroaryl, OC₁₋₆alkyl, SC₁₋₆alkyl, NH(C₁₋₆alkyl), N(C₁₋₆alkyl)(C₁₋₆alkyl), C(O)C₁₋₆alkyl, CO₂C₁₋₆alkyl, C(O)NHC₁₋₆alkyl, C(O)N(C₁₋₆alkyl)(C₁₋₆alkyl), PO(OC₁₋₆alkyl) (OC₁₋₆alkyl), SO₂C₁₋₆alkyl, SO₂NH(C₁₋₆alkyl), SO₂N(C₁₋₆alkyl)(C₁₋₆alkyl), NC₁₋₆alkylSO₂(C₁₋₆alkyl), and NHSO₂(C₁₋₆alkyl), and each alkyl, alkenyl, alkynyl, cycloalkyl, heterocycloalkyl, aryl, heteroaryl and alkylene group in R⁵ and R⁶ is optionally substituted with one or more substituents independently selected from halo, NH₂, NHC(NH)NH₂, C₁₋₆alkyl, OR¹¹, SR¹¹, NR¹²R¹³, C(O)R¹¹, CO₂R¹¹, CONR¹²R¹³, PO(OR¹⁴)(OR¹⁵), SOR¹¹ SO₂R¹¹, SO₂NR¹²R¹³, and NR¹²SO₂R¹¹, and each alkyl, alkylene, alkenyl and alkynyl is optionally fluorosubstituted.

In an embodiment, R⁵ and R⁶ are independently selected from halo, CHO, CN, NO₂, OH, SH, NH₂, CO₂H, C(O)NH₂, PO₃H, SO₂H, SO₂NH₂, NHSO₂H, C₁₋₆alkyl, C₂₋₆alkenyl, C₂₋₆alkynyl, OC₁₋₆alkyl, SC₁₋₆alkyl, NH(C₁₋₆alkyl), N(C₁₋₆alkyl)(C₁₋₆alkyl), C(O)C₁₋₆alkyl, CO₂C₁₋₆alkyl, C(O)NHC₁₋₆alkyl, C(O)N(C₁₋₆alkyl)(C₁₋₆alkyl), PO (OC₁₋₆alkyl)(OC₁₋₆alkyl), SO₂C₁₋₆alkyl, SO₂NH(C₁₋₆alkyl), SO₂N(C₁₋₆alkyl)(C₁₋₆alkyl), NC₁₋₆alkylSO₂(C₁₋₆alkyl), and NHSO₂(C₁₋₆alkyl), and each alkyl, alkenyl, alkynyl, and alkylene group in R⁵ and R⁶ is optionally substituted with one or more substituents independently selected from halo, NH₂, NHC(NH)NH₂, C₁₋₆alkyl, OR¹¹, SR¹¹, NR¹²R¹³, C(O)R¹¹, CO₂R¹¹, CONR¹²R¹³, PO(OR¹⁴)(OR¹⁵), SOR¹¹ SO₂R¹¹, SO₂NR¹²R¹³, and NR¹²SO₂R¹¹, and each alkyl, alkylene, alkenyl and alkynyl is optionally fluorosubstituted. In an embodiment, R⁵ and R⁶ are independently selected from H, F, Cl, CHO, CN, NO₂, OH, SH, NH₂, CO₂H, C(O)NH₂, PO₃H, SO₂H, SO₂NH₂, NHSO₂H, C₁₋₆alkyl, C₂₋₆alkenyl, C₂₋₆alkynyl, OC₁₋₆alkyl, SC₁₋₆alkyl, NH(C₁₋₄alkyl), N(C₁₋₄alkyl)(C₁₋₄alkyl), C(O)C₁₋₄alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), SO₂N(C₁₋₄alkyl)(C₁₋₄alkyl), NC₁₋₄alkylSO₂(C₁₋₄alkyl), and NHSO₂(C₁₋₄alkyl), and each alkyl, alkenyl, alkynyl, and alkylene group in R⁵ and R⁶ is optionally substituted with one or more substituents independently selected from F, Cl, NH₂, NHC(NH)NH₂, C₁₋₆alkyl, OR¹¹, SR¹¹, NR¹²R¹³, C(O)R¹¹, CO₂R¹¹, CONR¹²R¹³, PO(OR¹⁴)(OR¹⁵), SOR¹¹ SO₂R¹¹, SO₂NR¹²R¹³, and NR¹²SO₂R¹¹ and each alkyl, alkylene, alkenyl and alkynyl is optionally fluorosubstituted

In an embodiment, one of R⁵ and R⁶ is selected from C₁₋₆alkyl, OC₁₋₆alkyl, SC₁₋₄alkyl, NH(C₁₋₄alkyl), N(C₁₋₄alkyl)(C₁₋₄alkyl), C(O)C₁₋₄alkyl, CO₂C₁₋₄alkyl and the other is selected from F, Cl, CHO, CN, NO₂, OH, SH, NH₂, CO₂H, C(O)NH₂, PO₃H, SO₂H, SO₂NH₂, NHSO₂H, C₁₋₆alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), SO₂N(C₁₋₄alkyl)(C₁₋₄alkyl), NC₁₋₄alkylSO₂(C₁₋₄alkyl), and NHSO₂(C₁₋₄alkyl), and each alkyl group in R⁵ and R⁶ is optionally substituted with one or more substituents independently selected from halo, NH₂, NHC(NH)NH₂, C₁₋₆alkyl, OR¹¹, SR¹¹, NR¹²R¹³, C(O)R¹¹, CO₂R¹¹, CONR¹²R¹³, PO(OR¹⁴)(OR¹⁵), SOR¹¹ SO₂R¹¹, SO₂NR¹²R¹³, and NR¹²SO₂R¹¹, and each alkyl is optionally fluorosubstituted. In an embodiment, one of R⁵ and R⁶ is selected from C₁₋₆alkyl, OC₁₋₆alkyl, and SC₁₋₄alkyl and the other is selected from CO₂H, C(O)NH₂, PO₃H, SO₂H, SO₂NH₂, NHSO₂H, C₁₋₆alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), and SO₂N(C₁₋₄alkyl)(C₁₋₄alkyl) and each alkyl group in R⁵ and R⁶ is optionally substituted with one or more substituents independently selected from halo, NH₂, NHC(NH)NH₂, C₁₋₆alkyl, OR¹¹, SR¹¹, and NR¹²R¹³ and each alkyl is optionally fluorosubstituted. In an embodiment, one of R⁵ and R⁶ is C₁₋₄alkyl optionally substituted with one or more substituents independently selected from NH₂, NHC(NH)NH₂, OR¹¹, SR¹¹, and NR¹²R¹³ and the other is selected from CO₂H and C¹⁻⁶alkyl optionally substituted with one or more OR¹¹, and each alkyl is optionally fluorosubstituted. In an embodiment, one of R⁵ and R⁶ is C₁₋₄alkyl optionally substituted with one or more OR¹¹ and the other is CO₂H, and alkyl is optionally fluorosubstituted. In an embodiment, one of R⁵ and R⁶ is CH(OH)CH₃ and the other is CO₂H. In an embodiment, one of R⁵ and R⁶ is C₁₋₄alkyl and the other is C₁₋₄alkyl optionally substituted with one or more OR¹¹. In an embodiment, one of R⁵ and R⁶ is CH₃, and the other is CHCH(OH)₂.

In an embodiment, one of R⁵ and R⁶ are independently selected from C₃₋₁₆cycloalkyl, C₃₋₁₆heterocycloalkyl, C₆₋₁₆aryl, C₆₋₁₆heteroaryl, C₁₋₆alkyleneC₃₋₁₆cycloalkyl, C₁₋₆alkyleneC₃₋₁₆heterocycloalkyl, C₁₋₆alkyleneC₆₋₁₀aryl, and C₁₋₆alkyleneC₆₋₁₆heteroaryl, and the other is selected from F, Cl, CHO, CN, NO₂, OH, SH, NH₂, CO₂H, C(O)NH₂, PO₃H, SO₂H, SO₂NH₂, NHSO₂H, C₁₋₆alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), SO₂N(C₁₋₄alkyl)(C₁₋₄alkyl), NC₁₋₄alkylSO₂(C₁₋₄alkyl), and NHSO₂(C₁₋₄alkyl), and each alkyl, cycloalkyl, heterocycloalkyl, aryl, heteroaryl and alkylene group in R⁵ and R⁶ is optionally substituted with one or more substituents independently selected from halo, NH₂, NHC(NH)NH₂, C₁₋₆alkyl, OR^{11,} SR¹¹, NR¹²R¹³, C(O)R¹¹, CO₂R¹¹, CONR¹²R¹³, PO(OR¹⁴)(OR¹⁵), SOR¹¹ SO₂R¹¹, SO₂NR¹²R¹³, and NR¹²SO₂R¹¹. In an embodiment, one of R⁵ and R⁶ are independently selected from C₃₋₁₀cycloalkyl, C₃₋₁₀heterocycloalkyl, C₆₋₁₀aryl, C₅₋₁₀heteroaryl, C₁₋₆alkyleneC₃₋₁₀cycloalkyl, C₁₋₆alkyleneC₃₋₁₀heterocycloalkyl, C₁₋₆alkyleneC₆₋₁₀aryl, and C₁₋₆alkyleneC₅₋₁₀heteroaryl, and the other is selected from CO₂H, C(O)NH₂, PO₃H, SO₂H, SO₂NH₂, NHSO₂H, C₁₋₆alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), and SO₂N(C₁₋₄alkyl)(C₁₋₄alkyl) and each alkyl, cycloalkyl, heterocycloalkyl, aryl, heteroaryl and alkylene group in R⁵ and R⁶ is optionally substituted with one or more substituents independently selected from F, Cl, NH₂, C₁₋₆alkyl, OR¹¹, SR¹¹, and NR¹²R¹³. In an embodiment, one of R⁵ and R⁶ are independently selected from C₃₋₁₀cycloalkyl, C₃₋₁₀heterocycloalkyl, C₆₋₁₀aryl, C₅₋₁₀heteroaryl, C₁₋₄alkyleneC₃₋₁₀cycloalkyl, C₁₋₄alkyleneC₃₋₁₀heterocycloalkyl, C₁₋₄alkyleneC₆₋₁₀aryl, and C₁₋₄alkyleneC₅₋₁₀heteroaryl, and each cycloalkyl, heterocycloalkyl, aryl, heteroaryl and each alkylene group is optionally substituted with one or more substituents independently selected from F, Cl, NH₂, C₁₋₆alkyl, OR¹¹, SR¹¹, and NR¹²R¹³, and the other is selected from CO₂H, C(O)NH₂, PO₃H, SO₂H, SO₂NH₂, NHSO₂H, C₁₋₆alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), and SO₂N(C₁₋₄alkyl)(C₁₋₄alkyl) and each alkyl group is optionally substituted with one or more substituents independently selected from F, Cl, NH₂, C₁₋₆alkyl, OR¹¹, SR¹¹, and NR¹²R¹³. In an embodiment, one of R⁵ and R⁶ are independently selected from C₁₋₄alkyleneC₃₋₁₀cycloalkyl, C₁₋₄alkyleneC₃₋₁₀heterocycloalkyl, C₁₋₄alkyleneC₆₋₁₀aryl, and C₁₋₄alkyleneC₅₋₁₀heteroaryl, and each aryl, heteroaryl and each alkylene group is optionally substituted with one or more substituents independently selected from F, Cl, NH₂, C₁₋₆alkyl, OR¹¹, SR¹¹, and NR¹²R¹³, and the other is selected from CO₂H, C(O)NH₂, PO₃H, SO₂H, SO₂NH₂, NHSO₂H, C₁₋₆alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), and SO₂N(C₁₋₄alkyl)(C₁₋₄alkyl) and each alkyl group is optionally substituted with one or more substituents independently selected from F, Cl, NH₂, C₁₋₆alkyl, OR¹¹, SR¹¹, and NR¹²R^{13.}

In an embodiment, the aryl in R⁵or R⁶ is selected from phenyl, naphthyl and indanyl. In an embodiment the aryl in R⁵ or R⁶ is phenyl.

In an embodiment, the heteroaryl in R⁵ or R⁶ is selected from pyrrolyl, imidazolyl, oxazolyl, pyrazolyl, pyridinyl, pyrimidinyl, thiophenyl, indolyl, isoindolyl, quinolinyl, and isoquinolinyl.

In an embodiment, cycloalkyl in R⁵ or R⁶ is selected from cyclopropyl, cyclobutyl, cyclopentyl and cyclohexyl.

In an embodiment, the heterocycloalkyl in R⁵ or R⁶ is selected from azetidinyl, oxetanyl, tetrohydrofuranyl, tetrahydrothiophenyl, pyrrolidinyl, imidazolidinyl, pyrazolidinyl, thiazolidinyl, isothiazolidinyl, dioxolanyl, dithiolanyl, thianyl, piperidinyl, piperazinyl, tetrahydropyranyl, thiomorpholinyl, morpholinyl, and dioxanyl.

In an embodiment, R⁷ is selected from H or C₁₋₄alkyl, and alkyl is optionally fluorosubstituted. In an embodiment, R⁷ is selected from H, CH₃, CHF₂, or CF₃. In an embodiment, R⁷ is H.

In an embodiment, R¹¹, R¹², R¹³, R¹⁴, and R¹⁵ are independently selected from H and C₁₋₄alkyl; and alkyl is optionally fluorosubstituted.

In an embodiment, the compounds of Formula I are selected from compounds of the following Formulae:

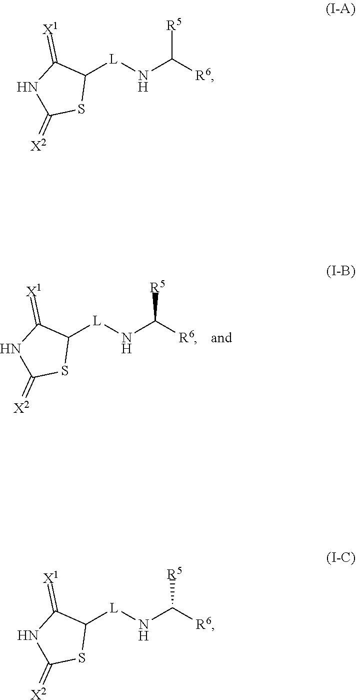

or a pharmaceutically acceptable salt and/or solvate thereof.

In an embodiment the pharmaceutically acceptable salt of compounds of Formula I and Formula II is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of Formula I and Formula II include, for example, those made with solvents that are pharmaceutically acceptable. Examples of such solvents include water (resulting solvate is called a hydrate) and ethanol and the like. Suitable solvents are physiologically tolerable at the dosage administered.

In embodiments of the present application, the compounds of Formula I and Formula II described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of Formula I and Formula II may also exist in different tautomeric forms and it is intended that any tautomeric forms which the compounds form, as well as mixtures thereof, are included within the scope of the present application.

The compounds of Formula I and Formula II may further exist in varying polymorphic forms and it is contemplated that any polymorphs, or mixtures thereof, which form are included within the scope of the present application.

### IV. Compositions of the Application

The present application includes a pharmaceutical composition comprising one or more compounds of the application or a pharmaceutically acceptable salt and/or solvate thereof and carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier.

In an embodiment, the pharmaceutical compositions are used in the treatment of any of the diseases, disorders or conditions described herein.

The compounds of the application are administered to a subject in a variety of forms depending on the selected route of administration, as will be understood by those skilled in the art. For example, a compound of the application is administered by oral, inhalation, parenteral, buccal, sublingual, nasal, rectal, vaginal, patch, pump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

In some embodiments, a compound of the application is administered parenterally. For example, solutions of a compound of the application are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, a compound of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein a compound of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, Pa., 1980, pp. 1530-1533 for further discussion of suppository dosage forms.

In some embodiments a compound of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

A compound of the application including pharmaceutically acceptable salts and/or solvates thereof is suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

### IV. Methods of Preparing the Compounds of the Application

Compounds of the present application or salts and/or solvates thereof, are available from commercial sources or can be prepared using methods known in the art.

The formation of a desired compound salt is achieved using standard techniques. For example, the neutral compound is treated with an acid or base in a suitable solvent and the formed salt is isolated by filtration, extraction or any other suitable method.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### EXAMPLES

The following non-limiting examples are illustrative of the present application.

### Example 1

### Cav3.2-USP5 ELISA Assay

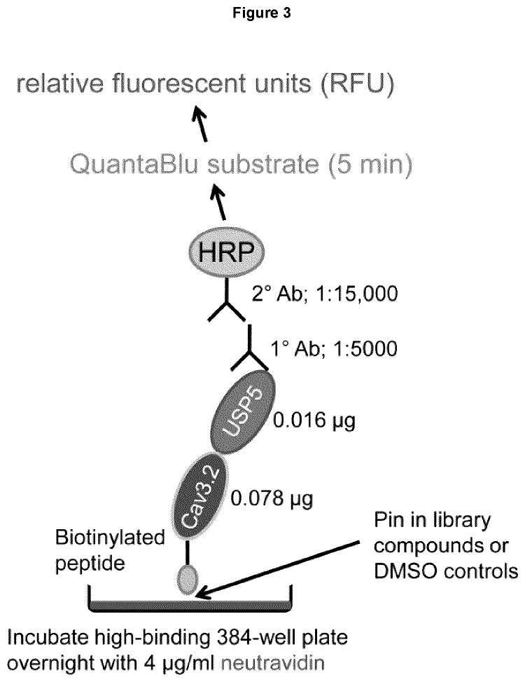

A high throughput ELISA assay was used to screen the exemplary compounds of the applications (FIG. 3). The ELISA is based on the binding of recombinant USP5 to immobilized Cav3.2 domain III-V linker peptides. Binding interactions were detected using USP5 antibodies, and a secondary antibody conjugated to horseradish peroxidase, leading to a fluorescence readout. The ELISA was converted into a high through-put format 384 well plate. Multiple compound libraries were screened for small organic molecules that disrupt the USP5—Cav3.2 interaction. A number of hits were identified at the standard screening concentration, and the dose dependence of hits was investigated. Select actives were subjected to a secondary biochemical assays based on co-immunoprecipitations of USP5 and Cav3.2 from mouse neuronal tissue. This resulted in confirmed hits from two classes of compounds related to gossypetin—one based on a flavonoid structure which was not further pursued, and another encompassed a thiazole scaffold with the two selected exemplary actives II-1 and II-2.

### Example 2

### Immunoprecipitation Assays

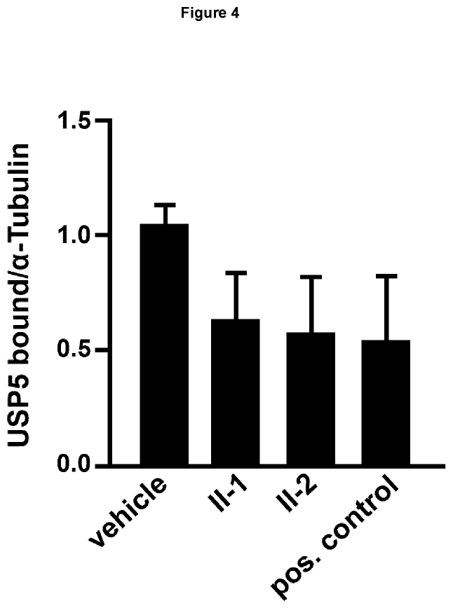

A co-immunoprecipitation assay was implemented as a secondary screen for actives derived from the compound library screen. Moause brain homogenate was prepared, and the tissue containing was incubated with a rabbit Cav3.2 polyclonal antibody to immunoprecipitate Cav3.2 channel complexes. The precipitates were run on a Western blot and probed with an antibody against USP5 (n=5). Band intensities for bound USP5 were quantified and normalized to alpha-tubulin (FIG. 4). 1 μM of exemplary compounds II-1 and II-2 were used in the incubation. 5 μM of a control compound identified in a previous library screen was used as a positive control.

### Example 3

### Formalin-Induced Pain Assay

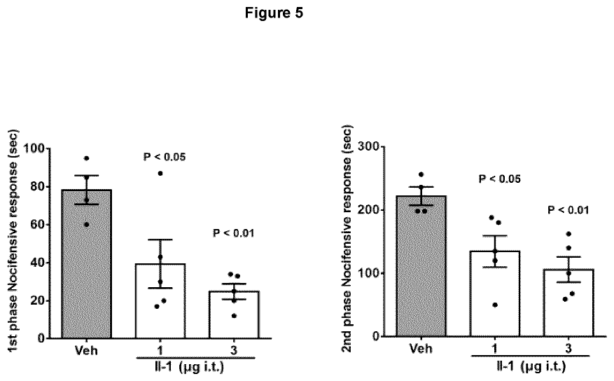

FIG. 5 shows the effect of exemplary compound II-1 on formalin induced nocifensive responses in phase I (left graph) and II (right graph) in mice. Formalin injection into the hindpaw mediates nocifensive responses (paw licking, biting). There is an initial phase that reflects an acute responses to formalin injection, followed by a quiescent period and a second phase of nocifensive behaviors that reflects inflammatory pain. The duration of the responses is reflective of the amount of pain. In both phases, the response time is shortened in a dose dependent manner by intrathecal delivery of exemplary compound II-1 in a dose-dependent manner. Hence, exemplary compound II-1 is analgesic in a mouse model of acute inflammatory pain. The same doses were then used in a chronic inflammatory pain assay (i.e. CFA).

### Example 4

### Complete Freund's Adjuvant (CFA) Model of Chronic Inflammatory Pain

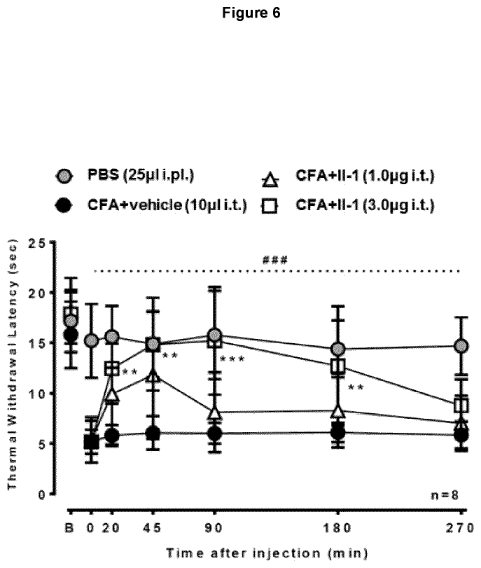

FIG. 6 shows the effect of exemplary compound II-1 on Complete Freund's Adjuvant (CFA) induced thermal hypersensitivity in mice. CFA injection into the hind paw leads to long lasting (weeks) mechanical and thermal hypersensitivity of the hind paw that is reflective of chronic inflammatory pain states. Thermal hypersensitivity is assessed by applying a radiant heat source to the hind paw of the animal and measuring the time between application of the heat source and the withdrawal of the paw. In CFA treated animals, the withdrawal latencies are dramatically shortened, and reflective of thermal hypersensitivity (i.e. thermal pain responses). The shortened latencies are reversed in a dose dependent manner by intrathecal delivery of exemplary compound II-1. Hence, exemplary compound II-1 is analgesic in mouse models of chronic inflammatory pain. Two asterisks indicate statistical significance at the 0.01 level, three asterisks indicate statistical significance at the 0.001 level relative to CFA+vehicle.

### Example 5

### Exemplary Compound II-1 has no Effect in Cav3.2 Null Mice

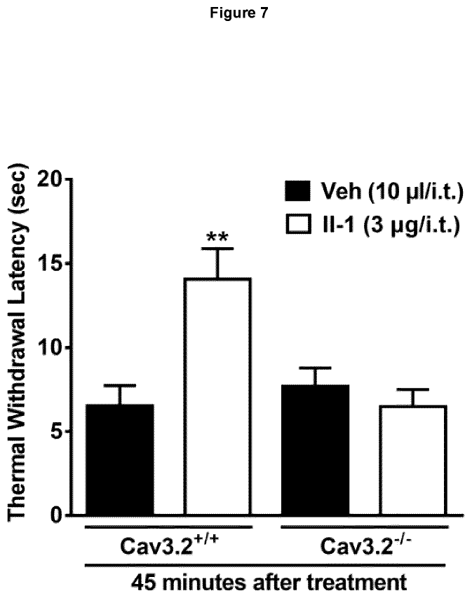

FIG. 7 compares the effects of exemplary compound II-1 on CFA mediated thermal hypersensitivity in wild type mice and in mice lacking Cav3.2 channels (45 minutes after administration of the compound). In wild type Cav3.2^{±/±} mice, exemplary compound II-1 reversed the CFA-induced reduction in thermal paw withdrawal latencies. Cav3.2^{-/-} mice have compensatory mechanisms that lead to increased thermal hypersensitivity in response to CFA even though Cav3.2 channels are absent. In these mice, exemplary compound II-1 is no longer able to reverse CFA induced thermal hypersensitivity. These data show that Cav3.2 channels are an essential targt5e of exemplary compound II-1 in the context of inflammatory pain and rule out analgesics actions of this compound via another molecular target.

### Example 6

### Exemplary Compound II-1 does not affect USPS enzymatic activity.

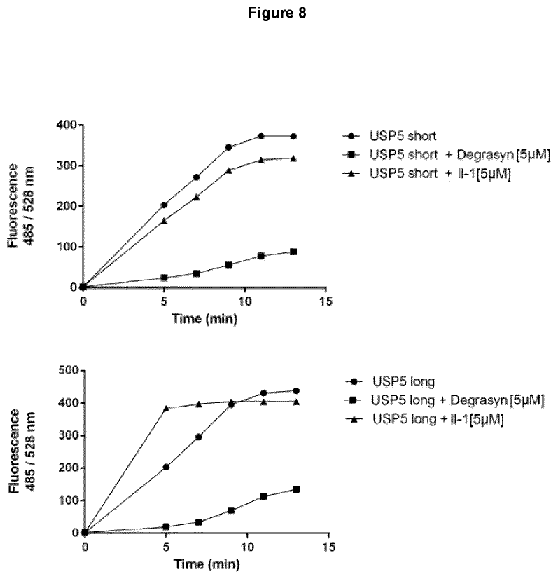

To rule out the possibility that exemplary compound II-1 might mediate its analgesic effects by altering the deubiquitinase (DUB) activity of USPS rather than by disrupting the interactions with the channel, a DUB assays was conducted (FIG. 8). Here, a ubiquitinated substrate (ubiquitin-rhodamine110-glycine) is incubated with recombinant long or short forms of USP5, and deubiquitination of the substrate as a function of time is measured via a fluorescence readout. Both the long form and the short form of USP5 lead to deubiquitination of the substrate, as evident from an increasing and plateauing fluorescence signal (528 nM emission in response to 485 nM excitation). The commercially available DUB inhibitor Degrasyn which is used as a positive control prevents this. Exemplary compound II-1 does not mediate DUB inhibition. It is possible to conclude therefore that exemplary compound II-1 does not act on enzymatic activity of USP5.

### Example 7

### Exemplary Compound II-1 Dose-Response Percent Inhibition in the Cav3.2-USP5 ELISA Assay.

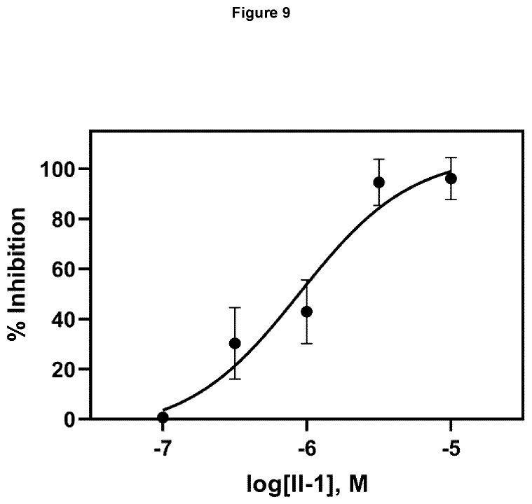

FIG. 9 shows a dose-response curve of exemplary compound II-1 on the percent inhibition of the Cav3.2-USP5 ELISA assay of Example 1. In the ELISA assay from Example 1, compound II-1 was added at varying concentrations, and the ability of II-1 to inhibit the USP5-Cav3.2 interaction examined. The percentage of inhibition as a function of drug concentration could be fitted using the Hill equation. These data show a dose dependence of the effect.

### Example 8

### Percent Inhibition of the Exemplary Compounds of Formula II in the Cav3.2-USP5 ELISA Assay.

Table 1 shows the percent inhibition of the exemplary compounds of Formula II in the Cav3.2-USP5 ELISA assay described in Example 1. These data show that there is a clear potential for structure-activity relationship depending on the groups attached to the thiazolidine core. Table 1

| Compound I.D | Chemical Structure | Percent Inhibition |
|---|---|---|
| II-1 | 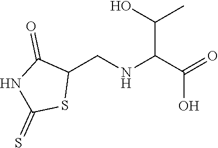 [SMILES: CC(O)C(NCC1SC(=S)NC1=O)C(=O)O] | 100 |
| II-2 | 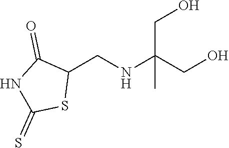 | 100 |
| II-3 | 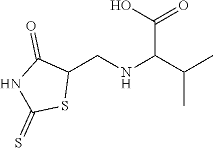 [SMILES: CC(C)C(NCC1SC(=S)NC1=O)C(=O)O] | 100 |
| II-4 | 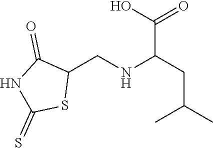 [SMILES: CC(C)CC(NCC1SC(=S)NC1=O)C(=O)O] | 100 |
| II-5 | 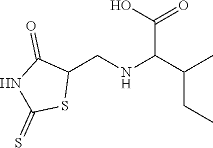 [SMILES: CCC(C)C(NCC1SC(=S)NC1=O)C(=O)O] | 86 |
| II-6 | 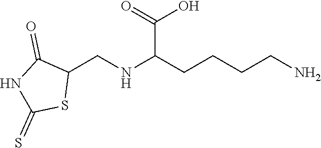 [SMILES: NCCCCC(NCC1SC(=S)NC1=O)C(=O)O] | 87 |
| II-7 | 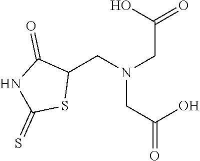 [SMILES: O=C(O)CN(CC(=O)O)CC1SC(=S)NC1=O] | 85 |
| II-8 | 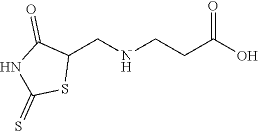 [SMILES: O=C(O)CCNCC1SC(=S)NC1=O] | 82 |
| II-9 | 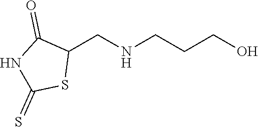 [SMILES: O=C1NC(=S)SC1CNCCCO] | 6 |
| II-10 | 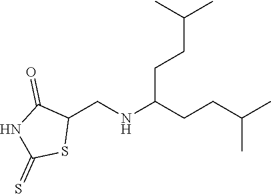 [SMILES: CC(C)CCC(CCC(C)C)NCC1SC(=S)NC1=O] | 28 |
| II-11 | 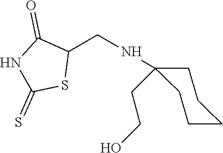 [SMILES: O=C1NC(=S)SC1CNC1(CCO)CCCCC1] | 75 |
| II-12 | 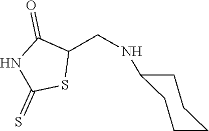 [SMILES: O=C1NC(=S)SC1CNC1CCCCC1] | 82 |

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE SPECIFICATION

A number of publications are cited herein. Full citations for these references are provided below. Each of these references is incorporated herein by reference in its entirety into the present disclosure, to the same extent as if each individual reference was specifically and individually indicated to be incorporated by reference.
1. Zamponi, G. W. 2016. Targeting voltage-gated calcium channels in neurological and psychiatric diseases. Nature Reviews Drug Discovery 15: 19-34.
2. Garcia-Caballero A, Zhang F X, Chen L, M'Dahoma S, Huang J, Zamponi GW., SUMOylation regulates USP5-Cav3.2 calcium channel interactions, Mol Brain. 2019 Aug 27;12(1):73. doi: 10.1186/s13041-019-0493-9. PMID: 31455361
3. Gadotti V M, Zamponi G W. Disrupting USP5/Cav3.2 interactions protects female mice from mechanical hypersensitivity during peripheral inflammation. Mol Brain. 2018 Oct 19;11(1):60. doi: 10.1186/s13041-018-0405-4. PMID: 30340616
4. Joksimovic S L, Joksimovic S M, Tesic V, Garcia-Caballero A, Feseha S, Zamponi G W, Jevtovic-Todorovic V, Todorovic S M. Selective inhibition of CaV3.2 channels reverses hyperexcitability of peripheral nociceptors and alleviates postsurgical pain, Sci Signal. 2018 Aug 28; 11 (545): PMID: 30154101
5. Stemkowski P L, Garcia-Caballero A, Gadotti V M, M'Dahoma S, Chen L, Souza I A, Zamponi G W., Identification of interleukin-1 beta as a key mediator in the upregulation of Cav3.2-USP5 interactions in the pain pathway, Mol Pain, 2017 Jan-Dec; 13: doi: 10.1177/1744806917724698
6. Stemkowski P, Garcia-Caballero A, Gadotti V M, M'Dahoma S, Huang S, Black SAG, Chen L, Souza I A, Zhang Z, Zamponi G W, TRPV1 Nociceptor Activity Initiates USP5/T-type Channel-Mediated Plasticity. Cell Rep. 2016 Dec 13;17(11):2901-2912. doi: 10.1016/j.celrep.2016.11.047. PMID: 27974205
7. Garcia-Caballero A, Gadotti V M, Chen L, Zamponi G W. A cell-permeant peptide corresponding to the cUBP domain of USP5 reverses inflammatory and neuropathic pain, Mol Pain, 2016 Apr 29;12: doi: 10.1177/1744806916642444. Print 2016. PM ID: 27130589
8. Gadotti V M, Caballero A G, Berger N D, Gladding C M, Chen L, Pfeifer T A, Zamponi G W. Small organic molecule disruptors of Cav3.2-USP5 interactions reverse inflammatory and neuropathic pain, Mol Pain, 2015 Mar 14;11:12. doi: 10.1186/s12990-015-0011-8. PMID: 25889575.
9 Garcia-Caballero A, Gadotti V M, Stemkowski P, Weiss N, Souza I A, Hodgkinson V, Bladen C, Chen L, Hamid J, Pizzoccaro A, Deage M, Francois A, Bourinet E, Zamponi G W, The deubiquitinating enzyme USP5 modulates neuropathic and inflammatory pain by enhancing Cav3.2 channel activity, Neuron. 2014 Sep 3;83(5):1144-58. doi: 10.1016/j.neuron.2014.07.036. PMID: 25189210

1. A method for inhibiting interactions between Cav3.2 T-type calcium channels (Cav3.2) and ubiquitin specific peptidase 5 (USP5) in a cell, either in a biological sample or in a patient, or a method of treating disease, disorder or condition that is treatable by inhibiting interactions between Cav.3.2 and USP5 comprising administering a therapeutically effective amount of one or more compounds of Formula II or a pharmaceutically acceptable salt and/or solvate thereof to the cell, or to a subject in need thereof, respectively, 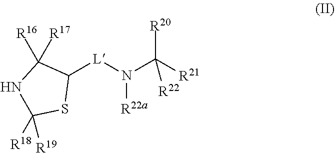
wherein
R¹⁶ and R¹⁸ are independently selected from H, halo, CN, NO₂, C₁₋₆alkyl, C₁₋₆haloalkyl, OR²³, SR²³, NR²⁴R²⁵, C(O)R²³, CO₂R²³, CONR²⁴R²⁵, SOR²³, SO₂R²³, and SO₂NR²⁴R²⁵;
R¹⁷ and R¹⁹ are independently selected from H and C₁₋₆alkyl; or
R¹⁶ and R¹⁷ are joined together to form =X³, and/or R¹⁸ and R¹⁹ are joined together to form =X⁴;
X³ and X⁴ are independently selected from O and S;
L′ is C₁₋₄alkylene;
R²⁰ and R²¹ are independently selected from H, halo, CHO, CN, NO₂, OH, SH, NH₂, CO₂H, C(O)NH₂, POSH, SO₂H, SO₂NH₂, NHSO₂H, C₁₋₁₀alkyl, C₂₋₁₀alkenyl, C₂₋₁₀alkynyl, C₃₋₁₆cycloalkyl, C₃₋₁₆heterocycloalkyl, C₆₋₁₀aryl, C₅₋₁₀heteroaryl, C₁₋₁₀alkyleneC₃₋₁₆cycloalkyl, C₁₋₁₀alkyleneC₃₋₁₆heterocycloalkyl, C₁₋₁₀alkyleneC₆₋₁₀aryl, C₁₋₁₀alkyleneC₅₋₁₆heteroaryl, NH(C₁₋₁₀alkyl), C(O)C₁₋₁₀alkyl, CP₂C₁₋₁₀alkyl, C(O)NHC₁₋₁₀alkyl, C(O)N(C₁₋₁₀oalkyl)(C₁₋₁₀alkyl), PO(OC₁₋₁₀alkyl)(OC₁₋₁₀alkyl), SO₂NH(C₁₋₁₀alkyl), SO₂N(C₁₋₁₀alkyl)(C₁₋₁₀alkyl), NC₁₋₁₀alkylSO₂(C₁₋₁₀alkyl), and NHSO₂(C₁₋₁₀alkyl), provided R²⁰ and R²¹ are not both H, or
R²⁰ and R²¹ together with the carbon to which they are attached form C₃₋₈cycloalkyl, and each alkyl, alkenyl, alkynyl, cycloalkyl, heterocycloalkyl, aryl, heteroaryl and alkylene group in R²⁰ and R²¹ is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁸, CONR²⁷R²⁸, PO(OR²⁹)(OR³⁰), SOR²⁸, SO₂R²⁸, SO₂NR²⁷R²⁸, and NR²⁷SO₂R²⁸;
R²² is selected from H, C₁₋₆alkyl and C₁₋₆alkyl and C₁₋₆alkyl substituted with one or more substituents selected from halo, NH₂, OH, SH, NH(C₁₋₆alkyl), N(C₁₋₆alkyl)₂, C(O)H, C(O)C₁₋₆alkyl, CO₂H, C(O)₂C₁₋₆alkyl, CONH₂, CONHC₁₋₆alkyl, CON(C₁₋₆alkyl)₂;
R^{22a} is selected from H, C₁₋₆alkyl and C₁₋₆alkyl substituted with one or more substituents selected from halo, NH₂, OH, SH, NH(C₁₋₆alkyl), N(C₁₋₆alkyl)₂, C(O)H, C(O)C₁₋₆alkyl, CO₂H, C(O)₂C₁₋₆alkyl, CONH₂, CONHC₁₋₆alkyl, CON(C₁₋₆alkyl)₂;
R²³, R²⁴, and R²⁵ are independently selected from H, C₁₋₆alkyl, C₂₋₆alkenyl and C₂₋₆alkynyl;
R²⁶, R²⁷, R²⁸, R²⁹, and R³⁰ are independently selected from H and C₁₋₆alkyl; and
each alkyl, alkylene, alkenyl and alkynyl is optionally fluorosubstituted.

2. (canceled)

3. The method of claim 1, wherein R¹⁶ and R¹⁸ are independently selected from H, F, Cl, CN, NO₂, C₁₋₃alkyl, C₁₋₃haloalkyl, OR²³, SR²³, NR²⁴R^{25,} C(O)R²³, CO₂R²³, C(O)NR²⁴R²⁵, SOR²³, SO₂R²³, and SO₂NR²⁴R²⁵ and each alkyl is optionally fluorosubstituted.

4. The method of claim 3, wherein R²³, R²⁴, and R²⁵ are independently selected from H and C₁₋₄alkyl and alkyl is optionally fluorosubstituted.

5. The method of claim 1, wherein R¹⁷ and R¹⁹ are independently selected from H and C₁₋₄alkyl, and alkyl is optionally fluorosubstituted.

6. The method of claim 1, wherein R¹⁶ and R¹⁷ are joined together to form =X³, and/or R¹⁸ and R¹⁹ are joined together to form =X⁴.

7. The method of claim 6, wherein X³ and X⁴ are both O or X³ and X⁴ are both S or one of X³ and X⁴ is O and the other is S.

8. (canceled)

9. The method of claim 1, wherein L′ is C₁₋₃alkylene, and alkylene is optionally fluorosubstituted.

10. (canceled)

11. (canceled)

12. The method of claim 1, wherein R²⁰ and R²¹ are independently selected from H, halo, CHO, CN, NO₂, OH, SH, NH₂, CO₂H, C(O)NH₂, PO₃H, SO₂H, SO₂NH₂, NHSO₂H, C₁₋₆alkyl, C₃₋₁₆cycloalkyl, C₃₋₁₆heterocycloalkyl, C₆₋₁₆aryl, C₅₋₁₆heteroaryl, C₁₋₆alkyleneC₃₋₁₆cycloalkyl, C₁₋₆alkyleneC₃₋₁₆heterocycloalkyl, C₁₋₆alkyleneC₆₋₁₀aryl, C₁₋₆alkyleneC₅₋₁₆heteroaryl, OC₁₋₆alkyl, NH(C₁₋₆alkyl), N(C₁₋₆alkyl)(C₁₋₆alkyl), C(O)C₁₋₆alkyl, CO₂C₁₋₆alkyl, C(O)NHC₁₋₆alkyl, C(O)N(C₁₋₆alkyl)(C₁₋₆alkyl), PO (OC₁₋₆alkyl)(OC₁₋₆alkyl), SO₂C₁₋₆alkyl, SO₂NH(C₁₋₆alkyl), SO₂N(C₁₋₆alkyl)(C₁₋₆alkyl), NC₁₋₆alkylSO₂(C₁₋₆alkyl), and NHSO₂(C₁₋₆alkyl), provided R²⁰ and R²¹ are not both H, and each alkyl, cycloalkyl, heterocycloalkyl, aryl, heteroaryl and alkylene group in R²⁰ and R²¹ is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁰), SOR²⁶, SO₂R²⁶, SO₂NR²⁷R²⁸, and NR²⁷SO₂R²⁶, and each alkyl and alkylene, is optionally fluorosubstituted.

13. (canceled)

14. The method of claim 1, wherein one of R²⁰ and R²¹ is selected from CO₂H, C₁₋₆alkyl, OC₁₋₆alkyl, NH(C₁₋₄alkyl), N(C₁₋₄alkyl)(C₁₋₄alkyl), C(O)C₁₋₄alkyl, CO₂C₁₋₄alkyl and the other is selected from H, F, Cl, CHO, CN, NO₂, OH, SH, NH₂, CO₂H, C(O)NH₂, PO₃H, SO₂H, SO₂NH₂, NHSO₂H, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), SO₂N(C₁₋₄alkyl)(C₁₋₄alkyl), NC₁₋₄alkylSO₂(C₁₋₄alkyl), and NHSO₂(C₁₋₄alkyl), and each alkyl group in R²⁰ and R²¹ is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁰), SOR²⁶, SO₂R²⁶, SO₂NR²⁷R²⁸, and NR²⁷SO₂R²⁶, and each alkyl is optionally fluorosubstituted.

15. (canceled)

16. The method of claim 1, wherein one of R²⁰ and R²¹ is selected from CO₂H and C₁₋₆alkyl optionally substituted with one or more OR²⁶, NH₂ and CO₂R²⁶, and the other is selected from H, CO₂H and C₁₋₆alkyl optionally substituted with one or more OR²⁶ and each alkyl is optionally fluorosubstituted.

17-30. (canceled)

31. The method of claim 12, wherein one of R²⁰ and R²¹ is selected from C₁₋₄alkyleneC₃₋₁₀cycloalkyl, C₁₋₄alkyleneC₃₋₁₀heterocycloalkyl, C₁₋₄alkyleneC₆₋₁₀aryl, and C₁₋₄alkyleneC₅₋₁₀heteroaryl, and each aryl, heteroaryl and each alkylene group is optionally substituted with one or more substituents independently selected from F, Cl, NH₂, C₁₋₆alkyl, OR²⁶, SR²⁶, and NR²⁷R²⁸, and the other is selected from CO₂H, C(O)NH₂, POSH, SO₂H, SO₂NH₂, NHSO₂H, C₁₋₆alkyl, CO₂C₁₋₄alkyl, C(O)NHC₁₋₄alkyl, C(O)N(C₁₋₄alkyl)(C₁₋₄alkyl), PO (OC₁₋₄alkyl)(OC₁₋₄alkyl), SO₂C₁₋₄alkyl, SO₂NH(C₁₋₄alkyl), and SO₂N(C₁₋₄alkyl)(C₁₋₄alkyl) and each alkyl group is optionally substituted with one or more substituents independently selected from F, Cl, NH₂, C₁₋₆alkyl, OR²⁶, SR²⁶, and NR²⁷R²⁸ and each alkyl and alkylene is optionally fluorosubstituted.

32. The method of claim 1, wherein R²⁰ and R²¹ together with the carbon to which they are attached form C₃₋₈cycloalkyl and the cycloalkyl is optionally substituted with one or more substituents independently selected from halo, NH₂, NHC(NH)NH₂, C₁₋₆alkyl, OR²⁶, SR²⁶, NR²⁷R²⁸, C(O)R²⁶, CO₂R²⁶, CONR²⁷R²⁸, PO(OR²⁹)(OR³⁶), SOR²⁶, SO₂R²⁶, SO₂NR²⁷R²⁸, and NR²⁷SO₂R²⁶, and each alkyl is optionally fluorosubstituted.

33. (canceled)

34. (canceled)

35. The method of claim 1, wherein R²² is selected from H, C₁₋₆alkyl and C₁₋₆alkyl substituted with one or more substituents selected from OH and CO₂H, and each alkyl is optionally fluorosubstituted.

36-41. (canceled)

42. The method of claim 1, wherein R²⁶, R²⁷, R²⁸, R²⁹, and R³⁰ are independently selected from H and C₁₋₄alkyl; and alkyl is optionally fluorosubstituted.

43. The method of claim 1, wherein the compound of Formula II is selected from compounds of the following Formulae: 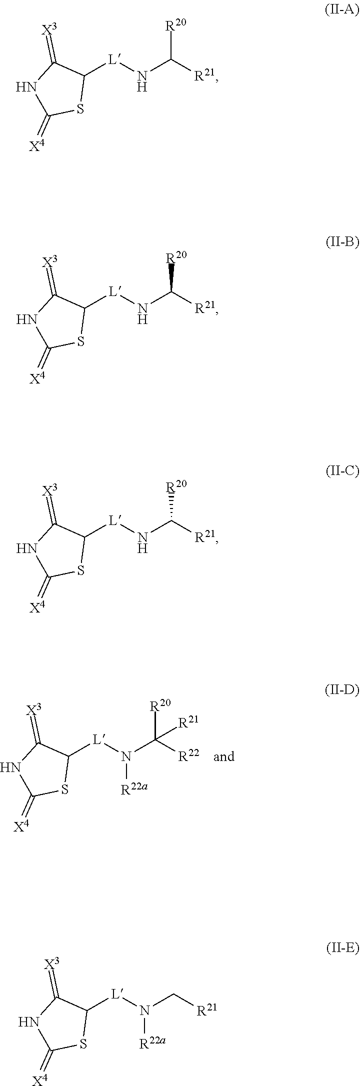
or a pharmaceutically acceptable salt and/or solvate thereof.

44. The method of claim 1, wherein the compound of Formula II is selected from the compounds listed below:

| Compound I.D | Chemical Structure |
|---|---|
| II-1 | 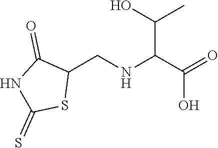 [SMILES: CC(O)C(NCC1SC(=S)NC1=O)C(=O)O] |
| II-2 | 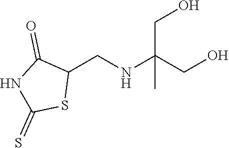 |
| II-3 | 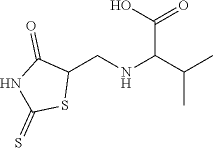 [SMILES: CC(C)C(NCC1SC(=S)NC1=O)C(=O)O] |
| II-4 | 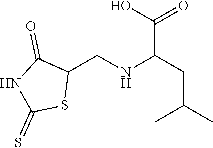 [SMILES: CC(C)CC(NCC1SC(=S)NC1=O)C(=O)O] |
| II-5 | 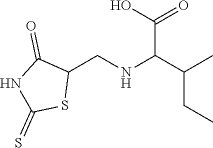 [SMILES: CCC(C)C(NCC1SC(=S)NC1=O)C(=O)O] |
| II-6 | 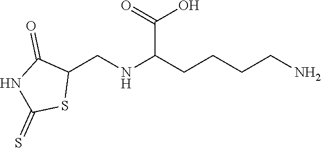 [SMILES: NCCCCC(NCC1SC(=S)NC1=O)C(=O)O] |
| II-7 | 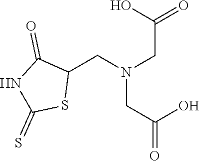 [SMILES: O=C(O)CN(CC(=O)O)CC1SC(=S)NC1=O] |
| II-8 | 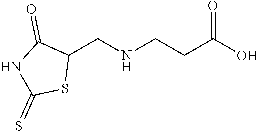 [SMILES: O=C(O)CCNCC1SC(=S)NC1=O] |
| II-9 | 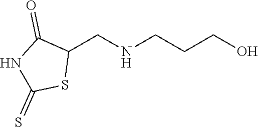 [SMILES: O=C1NC(=S)SC1CNCCCO] |
| II-10 | 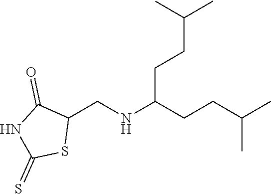 [SMILES: CC(C)CCC(CCC(C)C)NCC1SC(=S)NC1=O] |
| II-11 | 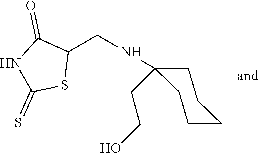 [SMILES: O=C1NC(=S)SC1CNC1(CCO)CCCCC1] |
| II-12 | 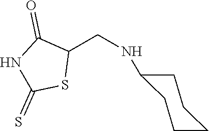 [SMILES: O=C1NC(=S)SC1CNC1CCCCC1] |

or a pharmaceutically acceptable salt and/or solvate thereof.

45. The method of claim 44, wherein the compound of Formula II is selected from the compounds listed below: 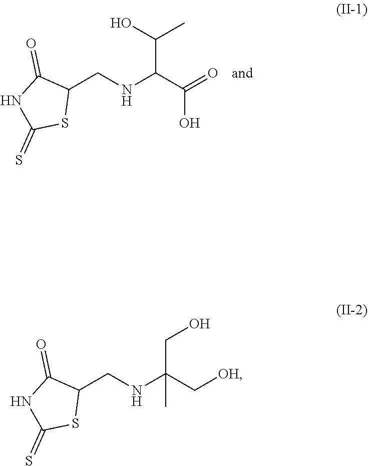 [SMILES: CC(O)C(NCC1SC(=S)NC1=O)C(=O)O ; CC(CO)(CO)NCC1SC(=S)NC1=O]
or a pharmaceutically acceptable salt and/or solvate thereof.

46. (canceled)

47. A method for inhibiting Cav3.2 deubiquitination in a cell, either in a biological sample or in a patient, or a method for treatng a disease, disorder or condition that is treatabe by inhibiting Cav3.2 deubiquitination comprising administering a therapeytically comprising administering an therapeutically effective amount of one or more compounds of Formula II as defined in claim 1 or a pharmaceutically acceptable salt and/or solvate thereof to the cell or to a subject in need thereof, respectively.

48. (canceled)

49. The method of claim 1, wherein the disease, disorder or condition is pain.

50-56. (canceled)

57. A method for inhibiting Cav3.2 function and/or expression by inhibiting interactions between Cav3.2 and USP5 in a cell, either in a biological sample or in a patient, or for traeting a disease, disorder or condition mediated or treatable by inhibiting CAV3.2 function and/or expression by inhibiting interactions between Cav3.2 and USP5 in a cell comprising administering a therapeutically effective amount of one or more compounds of Formula II as defined in claim 1 or a pharmaceutically acceptable salt and/or solvate thereof to the cell or to a subject in need thereof, respectively.

58. (canceled)
